(19) United States
(12) Patent Application Publication
(10) Pub. No.: US 2025/0229272 A1
(43) Pub. Date: Jul. 17, 2025

(54) Headspace Eliminating Microtiter Plate Lid

(71) Applicant: Agilent Technologies, Inc., Santa Clara, CA (US)

(72) Inventors: Tom Erik Pedersen, Cork (IE); James N. Hynes, Ovens, Cork (IE); Ryan McGarrigle, Glanmire, Cork (IE); Conn Carey, Ballygarvan, Cork (IE)

(21) Appl. No.: 18/849,119

(22) PCT Filed: Apr. 7, 2023
(86) PCT No.: PCT/US2023/017806

§ 371 Date: Sep. 20, 2024

Related U.S. Application Data
(60) Provisional application No. 63/328,894, filed on Apr. 8, 2022.

(51) Int. Cl. B01L 3/00

(52) U.S. Cl. CPC B01L 3/50853; B01L 2300/041; B01L 2300/0663; B01L 2300/0829; B01L 2300/12

(57) ABSTRACT

A microtiter plate assembly is disclosed that includes a base having a plurality of wells and a lid having a plurality of projections corresponding to the plurality of wells. Each projection contains a radial notch and a canted distal tip, providing bubble free sealing, reduced oxygen back-diffusion, and increased sensitivity even at small sample volumes.

### RELATED APPLICATIONS

The present application is the national stage entry of International Patent Application No. PCT/US2023/017806 having a filing date of Apr. 7, 2023, which claims priority to U.S. Provisional Application Ser. No. 63/328,894, filed on Apr. 8, 2022, both of which are incorporated herein in their entirety by reference hereto.

### BACKGROUND

Test tubes and microtiter plates are commonly used with oxygen-sensitive photoluminescent probes to measure and monitor aerobic activity of a sample by measuring and monitoring oxygen concentration within the tube or well. This requires sealing of the sample from fluid communication with the surrounding environment, often accomplished by providing an oil layer over the sample and interrogating the oxygen-sensitive photoluminescent probes in the sample through the oil layer. Use of an oil layer to seal off the sample provides the additional benefit of limiting the presence of gaseous headspace between the sample and the oil layer. Gaseous headspace trapped underneath the oxygen barrier layer is known to slow detection of changes in oxygen concentration due to the relatively large supply of oxygen available in such gaseous headspace.

While generally effective at sealing off the sample from direct fluid communication with the surrounding environment and limiting the presence of gaseous headspace underneath the oil layer, the oil layer is not a particularly effective oxygen barrier, is difficult to properly and consistently deploy, and is labor intensive. However, utilizing oil as a headspace eliminator requires an increased sample volume in order to avoid decreased optical pathlength (e.g. which may be caused by the oil contacting the bottom of the cell culture due to meniscus formation), which reduces sensitivity below that necessary for many measurement devices. For instance, such high sample volumes require correspondingly high seeding densities make it difficult to study cells at sub-confluent densities, which is further exacerbated when studying cells that have altered metabolism at confluence. Furthermore, oil as a headspace eliminator also requires a step of applying oil to each well, which creates potential for contamination and error, as well as eliminates the possibility of conducting post-assay testing.

Other devices, such as microtiter lids, have been proposed to allow increased visibility and control overspilling of samples. However, none of the proposed devices are designed for the purpose of metabolic interrogation in that they do not limit oxygen ingress to the sample nor do they adequately dissipate bubbles from the sample on sealing.

Therefore, while equipment has been proposed to eliminate the gaseous headspace in test tubes and microtiter plates, a need still exists for an improved system and method suitable for measuring and monitoring oxygen consumption, where both headspace and bubble formation is reduced.

### SUMMARY

The present disclosure is generally directed to a microtiter plate assembly that includes a base and a lid. The base includes at least one well, and the lid includes at least one translucent projection corresponding with at least one well. The at least one translucent projection extends from a proximal end adjacent to the lid to a distal end having a radial notch and a canted tip, the canted tip having an angle of about 5° or greater relative to a plane of the lid and a wall clearance is defined between a sidewall of the at least one projection and a sidewall of the at least one corresponding well. Furthermore, a radial notch clearance is defined between an apex of the radial notch and the sidewall of the corresponding well, wherein the wall clearance is 50% or less than the radial notch clearance.

The present disclosure is also generally directed to microtiter plate assembly that includes a base and a lid. The base includes a plurality of wells spaced apart in an array, where each well is adjacent to a second well, and the lid includes a plurality of translucent projections spaced apart in an array corresponding with the array of the plurality of wells, where each projection is adjacent to a second projection. In addition, the plurality of translucent projections extend from a proximal end adjacent to the lid to a distal end having a radial notch and a canted tip, the canted tip having an angle of about 5° or greater relative to a plane of the lid. Moreover, a height clearance is defined between a most distal point of the distal end of each projection and a well base of the respective well, where a percent variation of the height clearance between the projection and the well and the adjacent second projection and second well is about 10% or less.

In a further aspect, at least a portion of the assembly is formed from a substrate material having an oxygen transmission rate of about 600 cm³/m²/24 hours or less, and/or at least a portion of the assembly is coated with a polymeric or ceramic compound having an oxygen transmission rate of about 120 cm³/m²/24 hours or less. In one aspect, at least a portion of the assembly is coated with a Al₂O₃, SiO₂, silicon oxide, silicon nitride, silicon oxynitride, aluminum oxide, Al₂O₃ alternated with poly-acrylate, or a combination thereof.

In yet a further aspect, the radial notch has a radial cross section of about 0.7 mm² or greater, preferably wherein the radial notch has a radial cross section of about 1.25 mm² or greater. In one aspect, a radius of the radial notch has a length of about 5% to about 30% of a length of a radius of a cross section of the projection including the radial notch, at the proximal end of the projection, the distal end of the projection, or both the proximal end and the distal end of the projection. In yet a further aspect, the at least one projection has a proximal end radius and a distal end radius, wherein the distal end radius is about 2% to about 15% less than the proximal end radius

Moreover, in an aspect, the assembly is configured to measure oxygen consumption of fluid sample having a volume of 100 μL or less. In yet a further aspect, the assembly is configured to measure oxygen depletion of a sample having a seeding density of 250,000 cells or less in 45 minutes or less

In one aspect, the lid is in reversible association with the base. In another aspect, the lid is milled, extruded, injection molded, or a combination thereof. Furthermore, in an aspect, the assembly includes from about 1 to about 384 wells and about 1 to about 384 corresponding projections. In another aspect, the lid is a single piece. Additionally or alternatively, in an aspect, the lid is formed from a frame and one or more rows containing a plurality of translucent projections attached to the frame. In another aspect, the lid contains eight rows, each row containing twelve corresponding projections. Moreover, in an aspect, at least a portion of the assembly is formed from an acrylate polymer. Further, in another aspect, the lid can include a plurality of spacers.

The present disclosure is also generally directed to a method of measuring an oxygen depletion rate utilizing an assembly according to any one or more of the above discussed aspects. The method includes placing a sample and an oxygen-sensitive phosphorescent probe into at least one well, contacting the sample with the corresponding projection, and measuring the oxygen depletion with a fluorescence plate reader.

In one aspect, the method includes measuring extracellular acidification. Moreover, in an aspect, the oxygen-sensitive phosphorescent probe is a solid state sensor coated on the distal end of the projection, or wherein the oxygen-sensitive phosphorescent probe is a particle. In yet another aspect, the sample has a volume of 100 μL or less, preferably wherein the sample volume is 70 μL or less.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1A illustrates one aspect of a microtiter plate assembly according to the present disclosure;

FIG. 1B illustrates an aspect of a microtiter plate lid according to FIG. 1A;

FIG. 2A illustrates a top-down view of another aspect of a microtiter plate lid according to the present disclosure;

FIG. 2B illustrates the microtiter plate lid of FIG. 2A along cross-section B-B;

FIG. 2C illustrates a side perspective of the microtiter plate lid of FIG. 2A;

FIG. 2D illustrates a bottom-up view of the microtiter plate lid of FIG. 2A;

FIG. 2E illustrates a cross-section view of FIG. 2D along D-D;

FIG. 2F illustrates a perspective view of the microtiter plate lid of FIG. 2A;

FIG. 3A illustrates another aspect of a microtiter plate lid according to the present disclosure;

FIG. 3B illustrates a microtiter plate base corresponding to the microtiter plate lid of FIG. 3A;

FIG. 3C illustrates an aspect of a microtiter plate lid of FIG. 3A in releasable associate with the microtiter plate base of FIG. 3B;

FIG. 3D illustrates an aspect of a microtiter assembly according to the present disclosure;

FIG. 4A illustrates a further aspect of a microtiter plate assembly according to the present disclosure;

FIG. 4B is a zoomed in perspective of a distal end of a projection of FIG. 4A;

FIG. 4C is a zoomed in perspective view of a distal end of a projection according to the present disclosure;

FIG. 5A is a graph of re-oxygenation over time according to Example 1;

FIG. 5B is a graph of re-oxygenation over time according to Example 1;

FIG. 5C is a graph of re-oxygenation over time according to Example 1;

FIG. 6 is a graph of oxygen consumption over time according to Example 2;

FIG. 7 is a graph of oxygen consumption as a function of cell seeding density according to Example 3;

FIG. 8 is a graph of oxygen depletion over time according to Example 4;

FIG. 9 is a graph of pH calibration according to Example 5;

FIGS. 10A and 10B are graphs showing pH and oxygen depletion measured according to Example 5;

FIGS. 11A and 11B illustrate another aspect of a microtiter plate assembly according to the present disclosure;

FIG. 12 is a graph showing a comparison of the coefficient of variation of oxygen depletion measurements in the wells of a lid of the microtiter plate assembly of FIGS. 911A and 11B utilizing spacers having various thicknesses compared to a lid with no spacers;

FIG. 13 shows images to demonstrate cell viability for cells cultured in the plate assembly of the present disclosure for a control sample of culture media and for culture media containing tamoxifen after 1 hour and 24 hours;

FIG. 14 is a graph illustrating the oxygen consumption measured for cells grown in culture media containing tamoxifen after 1 hour and 24 hours;

FIG. 15 shows images taken for cells cultured in the plate assembly of the present disclosure for cells grown in a control sample of culture media and for cells grown in culture media containing rotenone, antimycin, and oligomycin;

FIG. 16 is a graph illustrating the oxygen consumption measured for cells grown in culture media containing rotenone, antimycin, and oligomycin compared to a control (untreated) media sample;

FIG. 17 shows a comparison of images taken when the lid of the plate assembly of the present disclosure is utilized compared to when no lid is utilized (open) when measuring JC-1;

FIG. 18 shows a comparison of images taken pre-treatment and post-treatment with FCCP and oligomycin using the plate assembly of the present disclosure;

FIG. 19 is a graph illustrating the oxygen consumption measured for cells grown in culture media containing FCCP and oligomycin compared to a cells grown in a control (untreated) culture media;

FIG. 20 is a graph illustrating kinetic JC-1 ratios measured for cells grown in culture media containing FCCP and oligomycin compared to a cells grown in a control (untreated) culture media;

FIG. 21 is a graph illustrating the ability to measure respiration for cells grown in suspension and treated with various compounds utilizing the plate assembly of the present disclosure;

FIGS. 22A-22D are images illustrating that suspension cells cultured in the plate assembly of the present disclosure can be loaded with JC-1 and imaged; and

FIG. 23 shows the JC-1 ratio calculated for the cells imaged in FIGS. 22A-22D.

Repeat use of reference characters in the present specification and drawings is intended to represent the same or analogous features or elements of the present invention.

### DETAILED DESCRIPTION

### Definitions and Test Methods

Oxygen concentration can be determined or indirectly interrogated by placing an oxygen-sensitive photoluminescent probe and a fluid test sample within a plurality of wells in a microtiter plate and ascertaining oxygen concentration within each well of the covered microtiter plate by exposing the oxygen-sensitive photoluminescent probe within each well to excitation radiation passed through the projection extending therein or the well bottom to create excited oxygen-sensitive photoluminescent material, measuring radiation emitted by the excited oxygen-sensitive photoluminescent material through the projection and the well bottom, and either converting the measured emission to a target-analyte concentration based upon a known conversion algorithm or measuring the probe/sensor signal in either intensity or lifetime modes. In one aspect, a suitable photoluminescent probe for such a measurement is MitoXpress® Xtra, available from Agilent Technologies. Instruments suitable for reading oxygen-sensitive photoluminescent probes within a cell sample are known and available from a number of sources, including the CLARIOstar plate reader from BMG Labtech GmbH of Ortenberg, Germany and the Synergy HTX from BioTek, an Agilent Technologies company. However, the photoluminescent material can also include an indicator dye incorporated in an oxygen permeable polymeric matrix, and it should be understood that electrochemical sensors can also be used to determine oxygen consumption. Oxygen Consumption Rate (OCR), which can also be referred to as oxygen depletion rate, as used herein can be calculated by sensing the metabolite (O₂) that is consumed from the media sample, which can then be reported in the form of a rate (change in analyte over time) or by measuring, or indirectly assessing, analyte concentration at a preselected timepoint (end point). Conversely, production of oxygen leading to an increase in oxygen concentration can also be determined by measuring the increase in oxygen over time.

Changes in oxygen consumption can be determined in sealed on unsealed systems. In one embodiment, the definition of OCR includes where oxygen consumption is not determined in a sealed system, e.g., a system allows oxygen back diffusion or substantial oxygen back diffusion to the sample, or where oxygen consumption is oxygen depletion in the sample corrected for oxygen back diffusion to the sample, or oxygen consumption is oxygen depletion without being corrected for oxygen back diffusion to the sample, or the oxygen consumption is determined in a sealed system, e.g., a system that does not allow oxygen back diffusion or substantial oxygen back diffusion to the sample, or oxygen consumption equals, or substantially equals, to oxygen depletion in the sample. Furthermore, in one embodiment, oxygen consumption is determined directly or indirectly, e.g., inferred from a measured oxygen gradient, e.g., within a test well, or by measuring oxygen at a preselected timepoint.

Extracellular Acidification Rate (ECAR), as used herein, can be determined using a basal or initial value for proton efflux for the cell sample, e.g., a value based upon a measurement of proton efflux for the cell sample made prior to formation of the reaction mixture and determining a proton efflux rate after formation of the reaction mixture. For instance, (ECAR), as used herein can be calculated by sensing the metabolite (H+) that is either being consumed or produced in the media sample, which can then be reported in the form of a rate (change in analyte over time) or by measuring analyte concentration at a preselected timepoint (end point). In one embodiment, changes in ECAR can be determined in a sealed or unsealed system.

With respect to data processing, in one aspect, a signal, such as a MitoXpress® signal, can be output in relative fluorescence units (RFU) and can be processed to adjust for the non-linear oxygen response of the MitoXpress® probe and any potential temperature equilibration in the assay. This is achieved by taking the natural log of the RFU signal and subtracting the natural log of the signal control at each time point. The oxygen calibration of the MitoXpress® probe has an exponential fit, which makes this a simple way of linearizing the signal. The level of temperature equilibration and drift in the probe signal is captured in the signal control and is accounted for through the subtraction. This ensures that a larger portion of the oxygen depletion trace is linear, resulting in a more robust data analysis and a reduced coefficient of variation (% CV) for the assay. This is particularly important for higher rates in RFU or lifetime scale, where few measurement points fall within a linear range of the curve. The signal can be further processed by normalizing to the first read of the assay to better assess and compare the compound responses.

As used herein, “translucence” or “translucent” means the light transmittance or optical density of a material which is in a range between transparent and opaque. Generally speaking, translucent materials allow light to pass through diffusely. A material that is translucent will allow a greater level of electromagnetic radiation in the visible spectrum to pass through it than an opaque or substantially opaque material but will allow a lower level of electromagnetic radiation in the visible spectrum to pass through it than a transparent or substantially transparent material. The translucent material can offer protection from phototoxicity while still allowing detection of the cells. A consequence is that cells cannot be observed from the top of the plate.

As used herein, the terms “about,” “approximately,” or “generally,” when used to modify a value, indicates that the value can be raised or lowered by 10%, such as, such as 7.5%, 5%, such as 4%, such as 3%, such as 2%, such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

### DETAILED DESCRIPTION

It is to be understood by one of ordinary skill in the art that the present discussion is a description of exemplary embodiments only and is not intended as limiting the broader aspects of the present disclosure.

Generally speaking, the present disclosure is directed to a microtiter assembly that includes a lid having a plurality of projections and a base having a plurality of reciprocating chambers (e.g., wells) corresponding to the respective projections, that eliminates headspace present in existing microtiter assemblies. Thus, in such a manner, the headspace eliminating microtiter lid of the present disclosure can also improve oxygen back diffusion and ingress while allowing a reduction in sample sizes and fill volumes. The present disclosure has unexpectedly found that a microtiter plate assembly having a plurality of projections extending longitudinally from a lid, where each projection has a combination of a radial notch and height clearance within specific ranges, combined with a canted distal end, allows ambient headspace oxygen to be reduced or eliminated, including completely eliminated, and also limits oxygen back diffusion (oxygen ingress) while also allowing reduced sample sizes.

Particularly, in one aspect, the present disclosure has found that by utilizing a specifically sized radial notch that extends in a lengthwise manner from the distal end to the proximal end of each projecting portion, air bubbles present in the sample chamber (e.g., a well) are evacuated while also preventing overspilling of the sample. In one aspect, the radial notch, which will be discussed in greater detail in regards to the figures below, has a radial cross-section of about 0.575 mm² or greater, such as about 0.585 mm² or greater, such as about 0.595 mm² or greater, such as about 0.6 mm² or greater, such as about 0.65 mm² or greater, such as about 0.7 mm² or greater, such as about 0.75 mm² or greater, such as about 0.85 mm² or greater, such as about 0.95 mm² or greater, such as about 1.00 mm² or greater, such as about 1.1 mm² or greater, such as about 1.2 mm² or greater, such as about 1.3 mm² or greater, such as about 1.4 mm² or greater, such as about 1.5 mm² or greater, such as about 1.6 mm² or greater, such as about 1.7 mm² or greater, such as about 1.75 mm² or greater, such as about 1.8 mm² or greater, such as about 1.9 mm² or greater, such as up to about 2 mm² or less. For instance, in one aspect, the radial notch has a radial cross-section from about 0.85 mm² to about 2.5 mm², such as from about 1 mm² to about 2.25 mm², or from about 1.25 mm² to about 2 mm², or any ranges or values therebetween.

Stated differently, in one aspect, the radial notch may have a cross-sectional area that is proportional to the cross-sectional area of the proximal end of the projection, the distal end of the projection, or both. For instance, the proximal end of the projection, the distal end of the projection, or both can define a cross-sectional area including the cross-sectional area defined by the radial notch. Thus, the radial cross-section of the radial notch may account for about 5% or more of the total cross-sectional area of the proximal end of each projection, such as about 8% or more, such as about 10% or more, such as about 15% or more, such as about 20% or more, such as about 25% or more, up to about 30% or less, such as about 5% to about 30%, such as about 8% to about 25%, or such as about 10% to about 20%, or any ranges or values therebetween. However, in one aspect, the above ranges reference a percent cross-sectional area occupied by the radial notch at a proximal end of the projection. As discussed above, the present disclosure has surprisingly found that when the radial notch has a radial cross section sized according to the above, the radial notch is able to allow escape of air bubbles from the sample without contributing to re-oxygenation or causing overspilling.

While the projection may have any radius as will be discussed in further detail below, in one aspect, the radial notch has a generally circular cross-sectional area. In such an aspect, a radius of the radial notch has a length relative to a length of the radius of the projection (at the proximal end, distal end, or both as defined above), from about 5% to about 30%, such as about 7.5% to about 27.5%, such as about 10% to about 25%, or any ranges or values therebetween.

Furthermore, the radial notch may have any cross-sectional shape, such as circular, elliptical, square, triangular, nodular or the like. However, in one aspect, the radial notch may have a circular cross section, such as circular or oval. Thus, in such an aspect, and as will be discussed in greater detail in regard to the figures below, the radial notch extends into the sidewall of each projection along the entire length of the respective projection, from distal end to proximal end, without penetrating or piercing the sidewall of the projection. Stated differently, the radial notch forms a portion of the sidewall of each projection, and does not extend into an interior portion of the projection. Therefore, it should be clear that any notch or channel formed by the radial notch is formed between the portion of the sidewall formed by the radial notch and a sidewall of a chamber or well conforming to the respective projection, and is not formed in an interior (e.g., hollow) of the projection.

In addition, as briefly mentioned, by using a radial notch having the above sizing and dimensions, the radial notch allows excess sample to be contained between the respective projection body and a side wall of the sample chamber or well without overspilling the chamber or well and contaminating adjacent chamber(s) or well(s). Furthermore, as will be discussed in greater detail below, in one aspect, the cross-sectional area of the projection (including the radial notch) may be larger at a proximal end than at a distal end, alone or in combination with a well that has a cross-sectional area that is larger at a proximal end than a distal end. In such a manner, a total overspill reservoir volume may be increased without sacrificing headspace elimination and evaporation prevention, for instance, in one aspect, the cross-sectional area of the proximal end of the projection is about 2% to about 15% larger than a cross-sectional area of the distal end of the projection, such as about 3% to about 12%, such as about 5% to about 9%, or any ranges or values therebetween.

Furthermore, in one aspect, the well may also have a cross-sectional area that is larger at a proximal end than a distal end. In such a manner, a total overspill reservoir volume may be increased without sacrificing headspace elimination and evaporation prevention, for instance, in one aspect, the cross-sectional area of the proximal end of the projection is about 3% to about 16% larger than a cross-sectional area of the distal end of the projection, such as about 4% to about 12.5%, such as about 5% to about 10%, or any ranges or values therebetween. Moreover, in one aspect, the well may have a proximal end cross-sectional area to distal end cross-sectional area ratio that is larger than a projection proximal end cross-sectional area to projection distal end cross-sectional area ratio. In such an aspect, both the well and the projection may have a taper as further described below, but at different taper percentages, (with the well having a larger taper), providing further reservoir volume and overspill prevention. Thus, the distal end of the projection, the proximal end of the projection, or both, is at least partially contacted by the sample or is fully immersed in the sample such that some sample is contained in a channel formed between the radial notch and the respective conforming chamber or well sidewall, as well as a lesser amount contained between a sidewall of the projection that does not contain the radial notch, and the respective chamber or well sidewall.

It should be noted that in one aspect, each projection has only one notch (whether for dissipation of oxygen or overspilling for other use). Namely, in one aspect when shaped and sized according to the above, only a single radial notch is needed to dissipate bubbles and provide an overspill containment area. However, in a further aspect, one or more of the projections can contain two notches, three notches, or four notches. In such an aspect, each notch may be shaped and sized as discussed herein. As will be discussed in greater detail below, in one aspect, at least one pillar has two notches formed symmetrically across a center line extending from the proximal end to the distal end of the pillar.

In one such aspect where the projection includes at least two notches, the distal end of the projection can contain a spotting surface that is generally parallel to a lower surface of the respective well or the plane of the lid. For instance, the spotting surface may have generally the same cross-sectional shape as the respective projection, and have a diameter of about 0.5 mm to about 4.5 mm, such as about 1 mm to about 4 mm, such as about 1.5 mm to about 3.5 mm, or any values or ranges therebetween. Furthermore, in one aspect where one or more of the lid, projection, base, or wells are formed to have a non-clear color, the spotting surface may be generally transparent or frosted.

However, while the sample can fully encircle or contact the distal end of at least some, or all, of the projections in one aspect, it should also be understood based upon the description of the method for determining oxygen consumption rate that the oxygen consumption rate is not determined based upon a visual inspection from above the sample. Instead, the oxygen consumption rate is determined utilizing a signal detector, such as a plate reader in one aspect, which measures data released from the photoluminescent material, which will be discussed in greater detail below. Therefore, in one aspect, the substrate material used to form some or all of the plurality of projections, lid, or base containing chambers or wells corresponding to the plurality of projections is formed from an translucent material, which is differentiated from a transparent material, by having limited viewing of the sample through the material while still allowing light to pass sufficient to enable probe interrogation, as defined above. Particularly, such a material can diffuse light while allowing the photoluminescent material to be measured according to the above method using a top-down plate reader, bottom-up plate reader, or others as known in the art. In one aspect, at least one of the base, the lid, and one or more projections can be translucent and have a black, white, or frosted appearance, or can be transparent.

Furthermore, the present disclosure has found that when the distal end of each longitudinal projection (e.g., the end of each projection opposite the end adjacent to the lid) is canted or sloped at a specific angle relative to a plane parallel to the lid (or well bottom) such that the highest side of the distal end (e.g., the side of the distal end that has a shorter length from the distal end to the lid) is adjacent to the radial notch, any air bubbles present in the sample are guided to the notch, facilitating removal of air bubbles. Therefore, in one aspect, the distal end is canted or sloped at an angle of about 5° or greater, such as about 7.5°, such as about 10° or greater, such as about 12.5° or greater, such as about 15° or greater, such as about 20° or greater, such as about 25° or greater, up to about 30° or less, relative to the lid or well bottom. For instance, in one aspect, the distal end is canted or sloped at an angle of about 3° to about 30°, such as about 3° to about 25°, such as about 5° to about 20°, or any ranges or values therebetween. Particularly, when angles according to the above are used for the distal end canted towards the radial notch, bubbles may be effectively removed from the sample while also allowing a highly sensitive oxygen consumption rate reading at small samples sizes.

Moreover, as may be understood, in an aspect contain one or more projections having two or more notches, the projection(s) may also contain two or more angled portions at the distal tip corresponding to the two or more projections. For example, in an aspect containing two projections, the canted distal tip can include two angled portions each having a highest side adjacent to a respective radial notch and having and each having a most distal point that meet at approximately the longitudinal center line. In such a manner, bubbles may be effectively removed to one or more notches for removal from the sample.

In addition, it was found that when a lid is used having projections according to the pending claims in combination with a canted distal end, a highly accurate height clearance (the distance between the most distal point of the distal and the lower surface of the chamber or well) is obtained. Due to this improved accuracy in height clearance, extremely low sample volumes can be used without losing functionality, which can also contribute to improved sensitivity to measurements, such as oxygen consumption rate, extracellular acidification, and other measurements, without disrupting cell growth or bubble removal. Namely, the canted distal end allows improved accuracy while eliminating headspace and therefore, oxygen, without contacting the lower wall of the chamber or well (unlike oil-in-headspace solutions). Thus, a microtiter assembly according to the present disclosure may have an average height clearance between the most distal point of each projection to the lower surface of the chamber or well when a stop tab of a lid is contacting the a base containing the plurality of chambers, of about 0.25 mm or less, such as about 0.20 mm or less, such as about 0.15 mm or less, such as about 0.1 mm or less, such as about 0.075 mm or less, such as about 0.05 mm or less, or any ranges or values therebetween. For instance, in one aspect, the microtiter assembly has an average height clearance of about 0.025 mm to about 0.25 mm, such as about 0.035 mm to about 0.15 mm, such as about 0.045 mm to about 0.1 mm, or any ranges or values therebetween.

Furthermore, as the lid assembly including the lid and above described projections allow for highly consistent height clearance, in one aspect, the height clearance may have a very low degree of variability, such that the variation in height clearance between adjacent projections/wells is about 10% or less, such as about 5% or less, such as about 2.5% or less, such as about 2% or less, such as about 1% or less. Particularly, the present disclosure has found that when a microtiter assembly is formed according to the above, increased sensitivity is achieved even with reduced sample sizes as well as highly consistent height clearance which further improve the sensitivity of the measurement.

For instance, a lid according to the present disclosure can be used to measure oxygen consumption rate, extracellular acidification rate (ECAR), and others as may be described herein, utilizing fill volumes of 100 μL or less, such as about 90 μL or less, such as about 80 μL or less, such as about 70 μL or less, such as about 60 μL or less, such as about 60 μL or less, such as about 50 μL or less, such as about 40 μL or less, such as about 30 μL or less, such as about 25 μL or less, such as about 20 μL or less, such as about 15 μL or less, such as about 10 μL or greater, such as about 15 μL or greater, or any ranges or values therebetween. For instance, in one aspect, the fill volume may be about 10 μL to about 100 μL, such as about 15 μL to about 90 μL, such as about 20 μL to about 80 μL, or any ranges or values therebetween.

Stated another way, lids according to the present disclosure allow lower seeding densities to be used while maintaining measurable results. For instance, in one aspect, the lid assembly according to the present disclosure can be utilized with seeding densities of about 100,000 cells or less, such as about 75,000 cells or less, such as about 50,000 cells or less, such as about 25,000 cells or less, such as about 12,500 cells or less, such as about 10,000 cells or less, such as about 7,500 cells or less, such as about 5,000 cells or less, such as about 2,500 cells or less, such as about 1,000 cells or greater, such as about 2,500 cells or greater, or any values or ranges therebetween. For instance, in one aspect, the seeding density is from about 2,500 cells to about 125,000 cells, such as about 6,500 cells to about 90,000 cells, such as about 10,000 cells to about 75,000 cells, or any ranges or values therebetween.

Namely, as mentioned above, by utilizing an assembly according to the present disclosure, oxygen ingress/back diffusion may be reduced while eliminating gaseous headspace from the well or chamber while also being compatible with small samples volumes. For instance, oil-in-headspace methods of minimizing oxygen contact require greater than 100 μL fill volumes in order to avoid oil contacting the bottom of the well in the center of the meniscus which destroys the optical path and rendering the sample unmeasurable. Conversely, assemblies according to the present disclosure allow a more disc-shaped view to be formed by the contact of the sample and the respective projection. Thus, very small sample volumes may be used while still reducing oxygen ingress/back diffusion, unlike prior approaches.

Thus, the assembly according to the present disclosure can provide benefits to fluid samples containing a variety of cell lines, for instance, due to increased sensitivity and reduced oxygen ingress/back diffusion. Namely, as discussed above, the assembly according to the present disclosure can be used with adherent cell lines as known in the art, in addition to suspension cell lines. As understood in the art, suspension cell lines can be immobilized on a coated plate (e.g. by use of a centrifuge and an immobilization coating such as PDL, PLL, collagen, and fibronectin for example, which will be discussed in greater detail below) and be assayed using any one or more of the methods described herein.

Furthermore, the assembly of the present disclosure can also be utilized with and provide benefits to 3D constructs. Examples of such constructs include spheroids (examples of which may be described in U.S. Provisional Patent Application No. 63/276,099, which is incorporated herein in its entirety) and/or organoids immobilized on coated plates, 3D culture systems utilizing scaffold or matrix materials (e.g. electrospun fibrous scaffolds, collagen, hydrogel, and Matrigel, including Lonza's RAFT™ 3D cell culture system), tissues and microtissues, and magnetic spheroids.

For instance, one such magnetic spheroid method that can benefit from small sample sizes includes a magnetic spheroid drive. As known in the art, magnetic spheroids can be measured using various methods in combination with low adhesion plates, magnetic particles, and magnets, for example, or may be localized (e.g. using poly lysine, a magnetic array, or the like). An example of one spheroid workflow, which is for example only, includes the Greiner workflow, where cells are magnetized, and formed into a spheroid utilizing a magnetic drive. After spheroid formation, the spheroids can be kept in the Greiner spheroid plate or moved to another suitable microplate. Regardless of the plate selected, a magnetic drive can be used to localize the spheroid while inserting the lid according to the present disclosure in order to orient the spheroid at the base of the well. Furthermore, in one aspect, the magnetic drive may be maintained during the assay to localize the spheroid.

Additionally or alternatively, as noted above, one or more of the projections may be hollow. Thus, in one aspect, the hollow core of one or more projections can contain a core magnet to aid in spheroid localization, allowing the lid to be placed without a magnetic drive. In one aspect, one or more of the projections contain a core magnet that is permanently affixed to the respective hollow. Conversely, in one aspect, the lid assembly according to the present disclosure may include a projection overlay that is disposed between the projections and the cover block. The projection overlay includes one or more magnetic spines that extend into the hollow of one or more projections in a releasable manner, and can be removed after spheroid localization or spheroid transport. In one such aspect, the projection overlay can include a number of magnetic spines corresponding to the number of projections, such that when the projection overlay is placed, each projection hollow is at least partially occupied with a magnetic spine.

Moreover, as may be understood by one having skill in the art, it should be understood that other fluid samples may benefit from a lid according to the present disclosure. For instance, the present disclosure can be used in conjunction with enzymes, isolated organelles, such as mitochondria, microscopic organisms, prokaryotes, eukaryotes, stem cells, cardiomyocytes, ex vivo samples embedded on microscope slides and the like. Nonetheless, while examples of various cell lines and samples have been provided, it should be understood that, in one aspect, any sample suitable for interrogation using a chamber or well according to the present disclosure that benefits from tailored sensitivity, reduced or controlled oxygen ingress/back diffusion, reduced sample volume (such as increased cell to volume ratio), or combinations thereof, may be used according to the present disclosure.

Furthermore, as may be understood by one having skill in the art, one or more culture coatings may be used on the plate, projection, lid, or combination thereof, based upon the fluid sample selected. For instance, as discussed above coatings for suspension cell lines may include a poly-D-lysine (PDL), poly-l-lysine (PLL) collagen, and fibronectin. Moreover, for spheroid assays, an ultra-low attachment coating, other common tissue culture coatings, or combinations thereof may be used. For instance, suitable ultra-low attachment coatings include BioFloat® Flex available from faCellitate and Lipidure® available from NOF Corporation which aid in avoiding unspecific surface binding of proteins and cells. However, it should be clear that other culture coatings may be used as known in the art.

Nonetheless, the lid according to the present disclosure is in reversible contact with the base and is therefore not permanently affixed to the base. Namely, each projection is in reversible association with the respective chamber or well. For instance, in one aspect, the stop tab of the lid can maintain contact with the base during the desired testing period using a variety of known methods, such as gravity, a weighted lid, or others. In one such aspect, a cover is applied over the lid, that releasably attaches to the base and applies a downward force to the lid.

To achieve the reversible attachment, the present disclosure has found that a specific wall clearance, defined as the distance between any portion of the side walls of each projection to the side wall of the chamber or well, is used to enable the lid to be removed while returning excess sample to the chamber or well. Namely, if too small of a wall clearance is used, the lid will not be removable, but if the wall clearance is too large, air bubbles become trapped between portions of the side wall that do not contain the radial notch, reducing the ability for air bubbles to be fully evacuated through the notch. Therefore, in one aspect, the assembly according to the present disclosure has a wall clearance of about 0.9 mm or less, such as about 0.8 mm or less, such as about 0.7 mm or less, such as about 0.6 mm or less, such as about 0.5 mm or less, such as about 0.4 mm or less, such as about 0.3 mm or less, such as about 0.2 mm or less, such as about 0.1 mm or less, such as about 0.05 mm or less, such as about 0.025 mm or less, or any ranges or values therebetween.

Stated differently, the wall clearance should be significantly smaller than the clearance by the radial notch in order to evacuate air bubbles through the notch instead of trapping air bubbles between the side wall of the projection and side wall of the corresponding well. Therefore, in one aspect, the radial notch defines a radial notch clearance defined as a distance between the point along the radial cross section that has the largest distance from the sidewall of the respective well (e.g. the apex of the radial notch), and the respective well sidewall, wherein the wall clearance is about 50% of the radial notch clearance or less, such as about 40% or less, such as about 30% or less, such as about 20% or less, such as about 10% or less, such as about 5% or less, such as about 2.5% or less, such as about 1% or less of the radial notch clearance.

Furthermore, as the lid assembly including the lid and above described projections allows for highly consistent wall clearance, in one aspect, the wall clearance may have a very low degree of variability, such that the variation in wall clearance between adjacent projections/wells is about 10% or less, such as about 5% or less, such as about 2.5% or less, such as about 2% or less, such as about 1% or less. However, it should be understood that, in one aspect, the lid may be permanently affixed to the base once placed in association with the base, such as an aspect where the assembly is not intended for reuse.

Regardless of the method used, a further advantage of the assembly of the present disclosure is that evaporation is limited due to the small wall clearance, while remaining reversible. Thus, unlike fixed or permanent lids, as well as samples utilizing oil to remove headspace, samples according to the present disclosure may undergo further testing after the cellular respiration measurements are complete. For instance, the assembly of the present disclosure would enable protein assays or fluorescent based assays to be completed after oxygen consumption rate testing, such as a post-assay immunofluorescence assays (including TOM 20 and LC-3, for example).

Nonetheless, it should also be understood that in addition to subsequent testing, additional simultaneous testing may be conducted in conjunction with the fluorescence based assay, which may also be referred to as “multiplexing”. Namely, as will be discussed in greater detail, as the assembly of the present disclosure is Society for Biomolecular Screening (SBS) compatible, and is therefore compatible with existing signal detectors for measuring additional target analytes (in addition to H+, CO, CO₂, O₂ discussed in greater detail herein) including calcium, ATP, NADP/NADHP, temperature, mitochondrial membrane potential, reactive oxygen species, or the like. Thus, while it should be understood by one having skill in the art that any additional testing may be conducted simultaneously that does not interfere with the target analyte may be multiplexed, in one aspect, additional testing can include stains (such as Hoechst and Calcein AM), florescent dyes, mitochondrial membrane potential (JC-1, e.g. TMRE/TMRM), reactive oxygen species (DHE), or combinations thereof.

Alternatively, the multiplexing analyte may require the use of a separate signal detector that does not interfere with the target analyte(s) discussed herein. The additional signal detector can have one or more detection modes, such as detection of absorbance, fluorescence intensity, luminescence, time-resolved fluorescence, fluorescence polarization, imaging, including florescence imaging, or combinations thereof. Plate readers having those detection modes are commercially available. Alternatively, in an aspect, the sensors can be interrogated from below the sensors, such as from below the lid of the apparatus; in such arrangements, the signal should be able to pass through the well and/or chamber so that it can be detected, for example, by having the well and/or chamber include a transparent material or translucent material, or being able to be interrogated from above without requiring a transparent material on a distal end of the projection, such as having one or more fiber optics passing through or contained within the lid and/or projections.

In addition, as briefly mentioned above, and as may be understood by one having skill in the art, the substrate material forming the base, lid, projections, or a combination thereof has increased contact with the sample due to the ability to overfill the chamber as well as the lack of oil barrier between the sample and the headspace or lid. Thus, as may be apparent to one having skill in the art, the assembly of the present disclosure allows the sample volume and/or fill volume, substrate material, and optional low oxygen transmission coating to be selected based upon the cell line to be studied. Namely, it is well known that different cells respire differentially, and may therefore deoxygenate the sample at different rates. For instance, in one aspect, cells that respire quickly, may require a larger fill volume or may have less sensitivity to the substate material or coating, whereas a cell line that is less active may benefit from a smaller sample volume and a coating or substrate material that limits back diffusion of oxygen. In one aspect, cell respiration is measured independent of fill volume. When measuring oxygen depletion over a set time with appropriate sensitivity, it has an optimum oxygen consumption:fill volume ratio. If the oxygen consumption is too high, then oxygen depletion is too fast to measure; if oxygen consumption is too low, then oxygen depletion is not measurable at all. Identifying a range in which oxygen depletion can be detected with measurable sensitivity provides a method of measuring cell respiration independent of fill volume. Nonetheless, in one aspect, the material used to form the substrate or to coat the substrate has an oxygen transmission rate of about 600 cm³/m²/24 hours or less, such as about 300 cm³/m²/24 hours or less, such as about 150 cm³/m²/24 hours or less, such as about 120 cm³/m²/24 hours or less, such as about 100 cm³/m²/24 hours or less, such as about 80 cm³/m²/24 hours or less, such as about 50 cm³/m²/24 hours or less, such as about 25, cm³/m²/24 hours or less, such as about 16 cm³/m²/24 hours or less, down to about 0 cm³/m²/24 hours or less or more measured at 23° C. and 0% RH according to ASTM D 3985.

In addition, in one aspect, as will be discussed in greater detail in the examples below, when low oxygen back diffusion is needed for sensitive samples, the material used to form the substrate or to coat the substrate has a low oxygen transmission rate of about 16 cm³/m²/24 hours or less, measured at 23° C. and 0% RH according to ASTM D 3985, such as about 15 cm³/m²/24 hours or less, such as about 12.5 cm³/m²/24 hours or less, such as about 10 cm³/m²/24 hours or less, such as about 7.5 cm³/m²/24 hours or less, such as about 5 cm³/m²/24 hours or less, such as about 2.5 cm³/m²/24 hours or less, such as about 1 cm³/m²/24 hours or less, such as about 0.5 cm³/m²/24 hours or less, or any ranges or values therebetween. For instance, in one aspect, the material has an oxygen transmission rate from about 0.01 cm³/m²/24 hours to about 20 cm³/m²/24 hours, such as about 0.05 cm³/m²/24 hours to about 17.5 cm³/m²/24 hours, such as about 0.1 cm³/m²/24 hours to about 15 cm³/m²/24 hours, or any ranges or values therebetween. Such low-oxygen transmission rates may aid in applications such as cancer research, as the necessary cell lines require low cell densities which are enabled by use of a lid according to the present disclosure as discussed herein.

For instance, in one aspect, any portion of the assembly may be formed from a substrate material having a variety of forms and compositions and may derive from naturally occurring materials, naturally occurring materials that have been synthetically modified, or synthetic materials. Examples of suitable substrate materials include, but are not limited to, nitrocellulose, glasses, silicas, teflons, metals (for example, gold, platinum, and the like), and ceramics (including aluminum oxide, silicon oxide, and the like), composites, and laminates thereof. Suitable substrate materials also include polymeric materials, including polysaccharides such as agarose (e.g., that available commercially as Sepharose®, from Pharmacia) and dextran (e.g., those available commercially under the tradenames Sephadex® and Sephacyl®, also from Pharmacia), polyacrylamides, polystyrenes, polyvinyl alcohols, copolymers of hydroxyethyl methacrylate and methyl methacrylate, polyesters, including poly(ethylene terephthalate) and poly(butylene terephthalate); polyamides, (such as nylons); polyethers, including polyformaldehyde and poly(phenylene sulfide); polyimides, such as that manufactured under the trademarks KAPTON (DuPont, Wilmington, Del.) and UPILEX (Ube Industries, Ltd., Japan); polyolefin compounds, including cyclic olefin polymers, ABS polymers, Kel-F copolymers, poly(methyl methacrylate), poly(styrene-butadiene) copolymers, poly(tetrafluoroethylene), poly(ethylenevinyl acetate) copolymers, poly(N-vinylcarbazole), polytetrafluoroethylene, polypropylene, polystyrene, polycarbonate, cyclo-olefin polymers (COP), such as sold under the brand name Zeonor® and Zeonex®, as well as cyclo-olefin copolymers (COC), such as sold under the brand name Topas®, and blends thereof, and the like. Certain polymeric materials that may be used for substrate materials include organic polymers that are either homopolymers or copolymers, naturally occurring or synthetic, crosslinked or uncrosslinked. A cover which may be disposed over the lid, may also be formed from the same types of materials as given herein for the substrate.

For instance, in one aspect, the substrate material for all or a portion of the assembly may be selected to be a material having a naturally low oxygen transmission rate, such as an acrylate polymer, which can be polymethylmethacrylate in one aspect, alone or further coated with a polymer having a very low oxygen transmission rate. In one aspect, any of the above substrate material can be used, but at least a portion of the substrate material is coated with a polymeric or ceramic compound, such as a glass compound or any compound suitable with low-temperature deposition processes, such as Al₂O₃ or SiO₂, atomic layer deposition (ALD), molecular vapor deposition, or plasma-enhanced chemical vapor deposition (PECVD), such as ceramics and polymers, including silicon oxide, silicon nitride, silicon oxynitride, aluminum oxide, mixed oxides, flexible glass (Al₂O₃ layers alternated with poly-acrylate layers on a PEN-based substrate, available as ClearProtect© 3D barrier coating from Antec), having a low oxygen transmission rate. Other surface treatments may be generally available include Actiplas, Aquacer, Carbocer, Lipocer, Plasmaclean, Silitec, or combinations thereof. Furthermore, in one aspect, all of a sample contacting portion of the plurality of projections are coated in a material having a very low oxygen transmission rate, alone or in combination with coating other portions of the assembly. Particularly, as will be discussed further in regards to the examples below, such a coating may further aid in preventing re-oxygenation of the sample after elimination of the headspace. Furthermore, it should be understood by one having skill in the art that additional reagents may be stored on the lid and/or projection, either by lyophilizing or solubilizing the reagent in a reagent substrate in order to further tune the assay. Exemplary reagent substrates include D-glucose-6-PO4, Pyruvic Acid, α-Keto-glutaric Acid, α-Keto-Butyric Acid, Ala-Gin, Sparker Malate Control, α-D-Glucose, citric acid, succinic acid, D,L-β-Hydroxy-Butyric Acid, L-Serine, Acetyl-L-Carnitine, γ-Amino-butyric acid, glycogen, D,L-α-Glycerol-PO4, D,L,-Isocitric Acid, Fumaric Acid, L-Glutamic Acid, L-Ornithine, Octanoyl-L-Carnitine, α-Keto-Isocaproic Acid, D-Glucose-1-PO4, L-Lactic Acid, cis-Aconitic Acid, L-Malic Acid, L-Glutamine, Tryptamine, Palmitoyl-D,L-Carnitine, L-leucine, and combinations thereof. Furthermore, exemplary reagents can include metabolic modulators, such as FCCP, Rotenone, Antimycin A, BAM15, and/or oligomycin, substrate catalyst, enzymes, including glucose oxidase, and exemplary inhibitors include Complex I Inhibitor Rotenone, Complex II Inhibitor Malonate, Complex III Inhibitor Antimycin A, Uncoupler FCCP, Ionophore K Valinomycin, Gossypol, Polymyxin B, Complex I Inhibitor Pyridaben, Complex II Inhibitor Carboxin, Complex III Inhibitor Myxothiazol, Uncoupler 2,4-Dinitrophenol, Calcium, CaCl2, Nordihydro-guaiaretic acid, Amitriptyline, Meclizine, Berberine, Alexidine, Phenformin, Diclofenac, Celastrol, Trifluoperazine, Papaverine, or combinations thereof. However, it should be understood that other reagent substrates, reagents, and inhibitors may be used as known in the art.

As discussed, in one aspect, the assembly according to the present disclosure is directed to a microtiter well plate assembly. Therefore, the plate cover and base may have 6, 8, 24, 96, 384, 1536 and so forth corresponding projections/wells as known in the art. However, in one aspect, the assembly of the present disclosure is particularly well suited for a microtiter plate assembly having 96 wells and corresponding projections.

Example aspects of the present disclosure are directed to an assembly and process for analyzing one or more biological constituents, including cellular parameters, contained in or associated with a biological material sample, such as a cell culture. The process and system utilize light detection and ranging components in a manner that not only efficiently takes readings, but also can take faster measurements than many conventional systems. However, it should be understood that, in one aspect, the probe and corresponding sensor may be selected to be any device capable of sensing and reporting changes in a target analyte, such as H+, CO, CO₂, O₂, or combinations thereof.

For instance, oxygen-sensitive photoluminescent probes capable of sensing and reporting the oxygen concentration of an environment in fluid communication with the probe are widely known. See for example, United States Published Patent Applications 2011/0136247, 2009/0029402, 2008/199360, 2008/190172, 2007/0042412, and 2004/0033575; U.S. Pat. Nos. 8,242,162, 8,158,438, 7,862,770, 7,849,729, 7,749,768, 7,679,745, 7,674,626, 7,569,395, 7,534,615, 7,368,153, 7,138,270, 6,989,246, 6,689,438, 6,395,506, 6,379,969, 6,080,574, 5,885,843, 5,863,460, 5,718,842, 5,595,708, 5,567,598, 5,462,879, 5,407,892, 5,114,676, 5,094,959, 5,030,420, 4,965,087, 4,810,655, and 4,476,870; PCT International Published Application WO 2008/146087; and European Published Patent Application EP 1134583, all of which are hereby incorporated by reference. Such optical sensors are available from a number of suppliers, including Presens Precision Sensing, GmbH of Regensburg, Germany, Oxysense of Dallas, Texas, USA, and Agilent Technologies of Cork, Ireland.

Methods and techniques for sensing of oxygen within a test tube or well of a microtiter plate using oxygen-sensitive photoluminescent probes are widely known as exemplified by WO2012/052068, US Pat. Appln. Pub 2013/0280751 and US Pat. Appln Pub. 2014/0147882, all incorporated herein by reference. These methods and techniques are suitable for use in determining oxygen concentration within a test tube or well sealed with an implement in accordance with the present invention.

However, while it has so far been discussed using a phosphorescent based sensor, it should be understood that optical sensors according to the present disclosure may also utilize solid-state, nanoparticulate, microparticulate, and/or magnetic sensors, or the like. For instance, solid state sensors may include one or more spots or films on the lid, base, projections, or combination thereof, where particle base sensors such as sensors coated onto beads may generally be in solution, in suspension, embedded within a suitable polymer, or reside in a sample container such as a well. Alternatively, in one aspect, particle based sensors can be loaded into cells or coated onto a surface, or embedded in a suitable polymer. Nonetheless, such sensors can include optical, O₂, pH, temperature, CO₂, or combinations thereof, as known in the art. For instance, in one aspect, the sensor is a solid state sensor coated on the distal end of the projection, a soluble sensor, a particulate sensor (that can be, e.g., a bead, magnetic bead, such as, residing in a sample container such as a well plate), a planar sensor (e.g., a film or a foil, such as on lid and microplate), or wherein the oxygen-sensitive phosphorescent probe is one or more sensor spots on the lid and microplate, or combination thereof (e.g., solid-state oxygen sensor on protrusion and soluble pH sensor in the media).

Furthermore, in one aspect, the sensor can be an electrochemical, or potentiometric sensors. Additionally or alternatively, electrodes may also be included in the well in order to measure electrical characteristics, including impedance. Notwithstanding the sensor selected, in one aspect, and as discussed above, it should be understood that the well or chamber may also contain one or more reference probes which generates a signal of known value for instrument calibration in the form of any of the sensors discussed above.

Regardless of the type of solid sensor selected, in one aspect for example only, the sensor may be embedded in a permeable medium, such as a permeable medium selected from hydrogel, silicone, and Matrigel. In some aspects, the sensor is attached at least one of the projections by solidifying or removing the medium (such as by drying, curing, cooling, evaporating or other technique). The solid-state sensor can be applied by dipping or spotting the distal end of at least one of projections in a mixture of a fluorescent indicator in a medium.

However, it should be appreciated that in certain aspects, the sensor can be spotted or dipped onto all or a portion of one or more of the projections. It should further be appreciated that in certain aspects, the sensor can be removably connectable to the body of one or more projections of the assembly. It should further be appreciated that in certain aspects, the sensors can be integrally formed with one or more projections. Integrally forming the sensors on one or a plurality of projections can be achieved by one or more techniques, such as vapor deposition, chemical coating, spin coating, dipping, and robotic spotting.

The assembly and processes according to example aspects of the present disclosure can be well suited to measuring constituents in all different types of samples, such as biological samples. In one aspect, for instance, the systems and processes according to example aspects of the present disclosure can be used to measure one or more constituents or a parameter related to the constituent in cellular material. The one or more constituents may be contained in a medium surrounding the cells or can be contained within the cells themselves. In some embodiments, the biological sample being tested may contain cellular material derived from cells, such as cellular organelles, mitochondria, or cellular extracts. Of particular advantage, the measurements can be completed in a label-free manner.

In one aspect, the cell sample is obtained or derived from a subject, such as a human or non-human animal. In one aspect, the subject is a mouse, which, in an aspect, has, or is at risk of having, a disorder. Nonetheless, in an aspect, the cell sample can include a primary cell, a cell isolated or harvested directly from a living tissue or organ, a cultured cell, and/or an immortalized cell. For instance, the cell sample can include a primary cell, or a cell isolated or harvested directly from a living tissue or organ, and then cultured ex vivo. In an aspect, the cell sample includes a cell that has been modified, e.g., genetically engineered for heterologous expression of a gene of interest, and/or genetically engineered for inhibition expression of a gene, such as cells from knock out mouse or CRISPR KO libraries. Nonetheless, in one aspect, the cell sample includes a stem cell or a cell derived from a stem cell. Nonetheless, regardless of the cell used, in one aspect, the cell sample includes a medium, e.g., a culture medium or a growth medium, where the cell can be disposed in the medium. Furthermore, as would be understood, in one aspect, the cell sample comprises a plurality of cells, e.g., a plurality of cells described herein.

The cells being tested can comprise any suitable cell sample, including but not limited to cultured cells, primary cells, human cells, neurons, T cells, B cells, epithelial cells, muscle cells, stem cells, induced pluripotent stem cells, immortalized cells, pathogen-infected cells, bacterial cells, fungal cells, plant cells, archaeal cells, mammalian cells, bird cells, insect cells, reptile cells, amphibian cells, and the like. The cells being tested may also comprise three-dimensional cell samples, such as tissue samples, cell spheroids, biopsied samples, cell scaffolds, organs-on-a-chip, and the like. Examples of parameters that may be measured and are related to the above cell functions include carbon dioxide concentration, oxygen concentration or oxygen partial pressure, calcium ions, hydrogen ions, and the like. However, in one aspect, the measured parameter is oxygen concentration, such as oxygen consumption. Through these tests, one can gain an understanding of what drives cell phenotype and function and/or an accurate picture of the cellular environment or microenvironment.

The assembly and process according to example aspects of the present disclosure can be used to measure live cell metabolic data, or (micro) environmental conditions of any viable cell. The cellular material being tested, for instance, can comprise bacteria cells, fungus cells, yeast cells, prokaryotic cells, eukaryotic cells, and the like. Cells that can be tested include mammalian cells including animal cells and human cells. Particular cells that can be tested include cancer cells, immune cells, immortal cells, primary cells, induced pluripotent stem cells, cells infected with viral or bacterial pathogens, and the like.

For example, in one aspect, the assembly and process according to example aspects of the present disclosure can be used to assist in immunotherapy. Immunotherapy is a type of treatment that bolsters a patient's immune system for fighting cancer, infections, and other diseases. Immunotherapy processes, for instance, can include adoptive cell based therapies, such as the production of T cells, Natural Killer (NK) cells, monocytes, macrophages, combinations thereof and the like. During T cell therapy, for instance, T cells are removed from a patient's blood. The T cells are then sent to a bioreactor and expanded or cultivated. In addition, the T cells can be changed so that they have specific proteins called receptors. The receptors on the T cells are designed to recognize and target unwanted cells in the body, such as cancer cells. The modified T cells are cultivated in a bioreactor to achieve a certain cell density and then supplied to a patient's body for fighting cancer or other diseases. T cell therapy can also be referred to as adoptive T cell therapy or T-cell transfer therapy, one example of which is referred to chimeric antigen receptor (CAR) T cell therapy. The use of T cells for adoptive T cell therapy or T-cell transfer therapy has recently proliferated due to great success in combating blood diseases. In some embodiments, aspects of the present invention may be used to monitor the health of T cells used in adoptive T cell therapy or T-cell transfer therapy. In some embodiments, aspects of the present invention may be used to monitor T cell activation, T cell exhaustion, T cell metabolism including of starting material and modified products, and the like.

NK cells are a type of cytotoxic lymphocyte that can seek out and destroy infected cells within the body. NK cells can display very fast immune reaction responses. Consequently, the use of NK cells in anticancer therapy has grown tremendously in interest and popularity. There is only a limited number of NK cells in the blood of a mammal, however, requiring that NK cells be grown to relatively high cell densities within bioreactors.

The culturing of cells, such as T cells, NK cells, or other mammalian cells, typically requires a somewhat complex process from inoculation to use in patients. The assembly and process of the present disclosure can be used to monitor cell metabolism during any point in the culturing process to ensure that the cells are healthy, and/or have the desired metabolic phenotype, and that the media in which the cells are growing contains an optimized level of nutrients. The system and process, for instance, can be used to make adjustments for assuring the metabolic fitness of the cells as they are growing.

In addition to immune cells, the metabolism of cancer cells can also be monitored for providing an understanding of which nutrients fuel the cancer cells. For example, the assembly and process according to example aspects of the present disclosure can reveal mechanisms or components that impact the metabolism of the cancer cells for inhibiting growth. The assembly and process according to example aspects of the present disclosure can also be used to determine the speed at which the cancer cells may proliferate. The system and process of the present disclosure is also well suited for use in toxicology. For instance, the process and assembly of the present disclosure can be used to detect mitochondrial liabilities among potential therapeutics. The risk of mitochondrial toxicity, for instance, can be assessed with high specificity and sensitivity. In this manner, the mechanism of action of some mitochondrial toxicants can be determined.

The systems and processes according to example aspects of the present disclosure can also be used to assist in treating obesity, diabetes, and metabolic disorders by aiding in the discovery of relevant therapies. For instance, the process and system can be used to measure functional effects of genetic changes to metabolic pathway components. Nutrients used in healthy and diseased cell models can be examined. Further, fatty acid oxidation and glycolysis can be assessed in different cell types, including stem cells.

Nonetheless, aspects of the present disclosure may be further understood according to the discussion of the following figures.

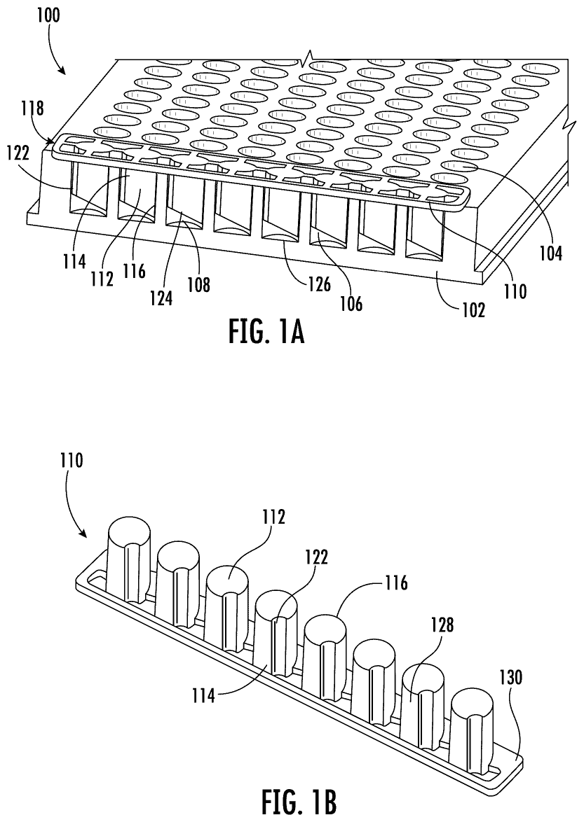

Referring to FIGS. 1A and 1B, an aspect of a base and lid for use in the assembly according to the present disclosure is illustrated. Namely, as shown in FIG. 1A, a microtiter assembly 100 is illustrated having a base 102 having a plurality of wells 104 having at least one sidewall 106 (shown more clearly in FIG. 1B) which each define a cavity 108. Furthermore, FIG. 1A shows a lid 110 having a plurality of projections 112 which extend longitudinally from a proximal end 114 to a distal end 116 in a generally perpendicular manner from the lid 110. While shown more clearly in FIG. 1B, each projection 112 has a radial notch 122 which extends from the proximal end 114 to the distal end 116 of each projection 112. Furthermore, each projection 112 has a canted distal tip 124. Particularly, as discussed above, the canted distal tip 124 is angled according to the above discussed ranges relative to the plane of the well base 126 and/or lid 110.

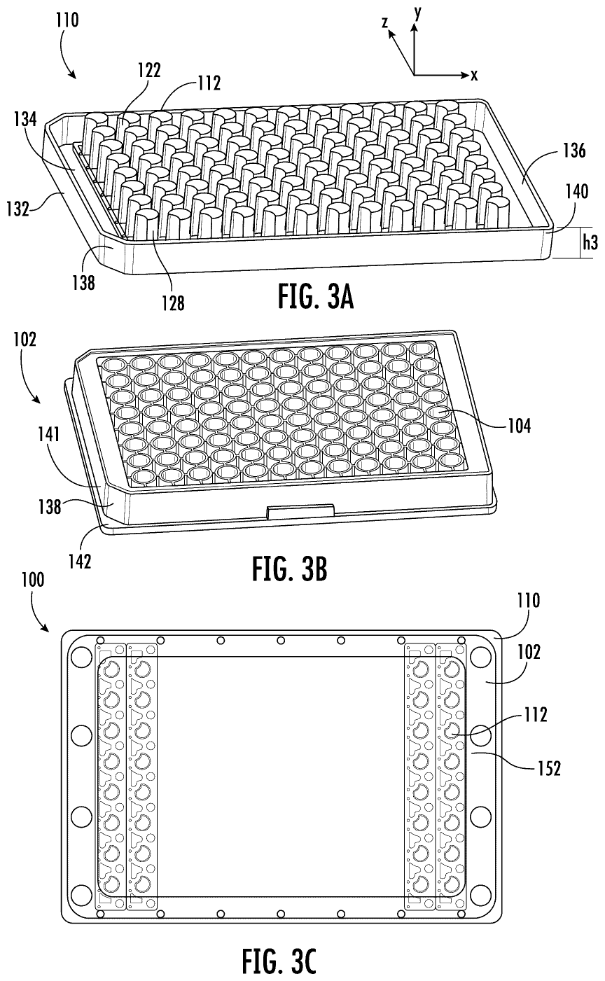

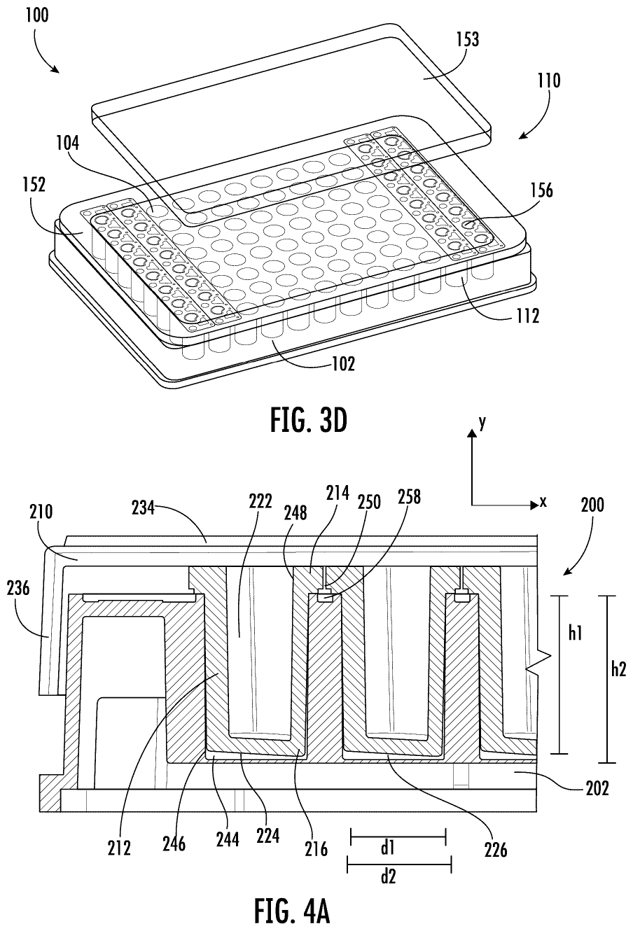

As shown in FIG. 1A, a first row 118 of wells 102 have the respective cavities 108 at least partially occupied with the corresponding projection 112. While FIG. 1A illustrates an assembly having only a single row 118 of projections 112 attached to a lid 110, it should be understood from the above that the lid 110 can extend across the entire assembly and contain a corresponding number of projections 112 as the number of wells 104 in the respective base 102. Furthermore, in one aspect, each lid 110 may contain one or more strips containing projections 112 where each strip may be attached on one or more ends to a frame (shown more clearly in FIG. 3D below). For instance, in regard to FIG. 1A, eleven (11) additional strips with corresponding projections 112 can be attached to a frame such that each well 104 is occupied by a corresponding projection 112 to form a full ninety-six (96) well microtiter plate 100.

Referring next to FIG. 1B, a lid 110 is shown separately from the assembly 100 with the projections 112 extending from a proximal end 114 to a distal end 116, each having a radial notch 122 extending into the at least one sidewall 128 of each projection. As shown in FIGS. 1A and 1B, the projections 112 are connected to a support 130. Furthermore, while only a single row in shown in FIG. 1B, it should be understood that the support 130 may be used to connect multiple lids 110 to a frame (not shown in FIGS. 1A and 1B).

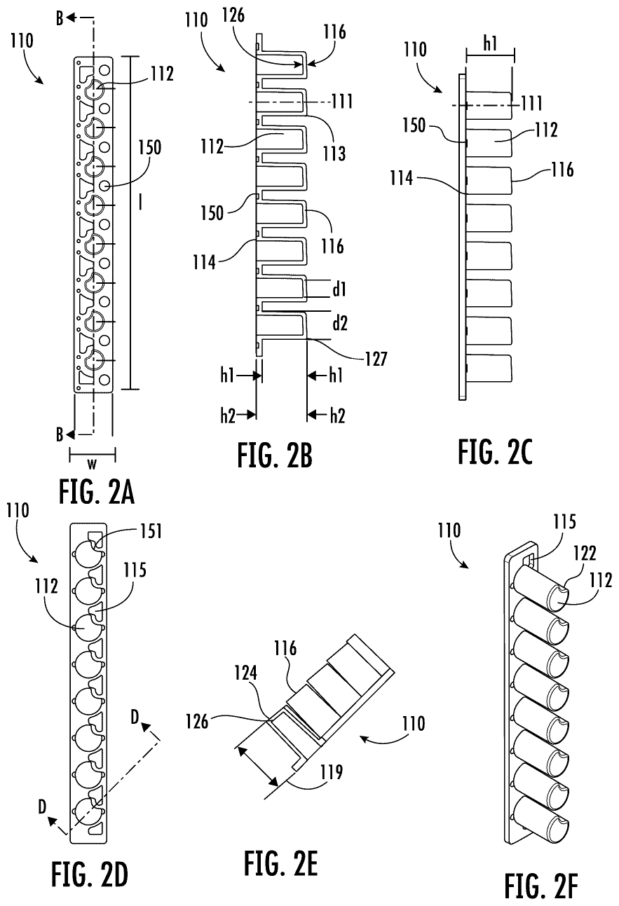

Similarly, FIGS. 2A-2F illustrate another aspect similar to FIGS. 1A and 1B, where lid 110 is in the form of a strip of eight projections 112. FIG. 2A shows a bottom-up view of the lid 110. As shown in FIG. 2A, the lid 100 in such an aspect can have a width w of about 12 mm or less, such as about 11 mm or less, such as about 10 mm or less, such as about 9 mm or less, such as about 5 mm or more, such as about 6 mm or more, such as about 7 mm or more, such as about 8 mm or more, or any ranges or values therebetween. For instance, in one aspect, the lid 110 has a width w from about 5 mm to about 12 mm, such as about 6 mm to about 11 mm, such as about 7 mm to about 9 mm, or any ranges or values therebetween.

Furthermore, as shown in FIG. 2A, which shows the apex 151 of the radial notch more clearly the lid 110 can have a length/of about 100 mm or less, such as about 95 mm or less, such as about 90 mm or less, such as about 85 mm or less, such as about 80 mm or less, or such as about 60 mm or more, such as about 65 mm or more, such as about 70 mm or more, such as about 75 mm or more, or any ranges or values therebetween. For instance, in one aspect, the lid 110 has a length of about 60 mm to about 100 mm, such as about 65 mm to about 90 mm, such as about 70 mm to about 85 mm, or any ranges or values therebetween.

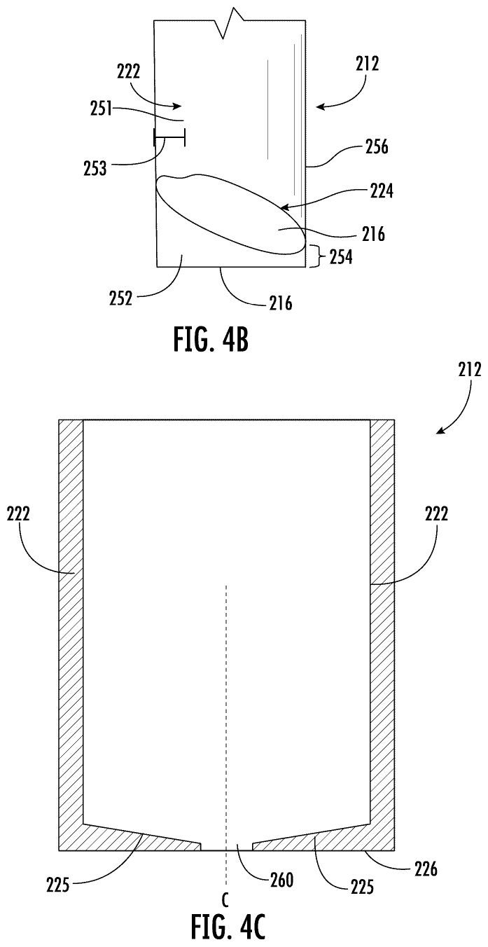

Similarly, referring to FIGS. 2B (which is a view along cross-section B-B of FIG. 2A) and 2C, in one aspect, the projections 112 have a height h1 from the stop tab 150 to a distal end 116 and a diameter d1, and the lid 110 has a height h2 from a stop tab receiving portion (not shown/shown more clearly in FIG. 4A) to a well base 126, and a diameter d2. Using these dimensions, the volume of the respective well 104 and the volume of the corresponding projection 112 can be determined to determine the fluid volume displaced by the lid 110. Furthermore, as shown in FIGS. 2B and 2C, the heights h1 and/or h2 can be measured from the center line 111 of each projection.

For instance, in one aspect, h1 is from about 15 mm or less, such as about 14 mm or less, such as about 13 mm or less, such as about 12 mm or less, such as about 11.5 mm or less, such as about 7 mm or greater, such as about 8 mm or greater, such as about 9 mm or greater, such as about 10 mm or greater, such as about 11 mm or greater, or any ranges or values therebetween. For instance, in one aspect, the lid 110 has a height h1 from about 7 mm to about 15 mm, such as about 9 mm to about 13 mm, such as about 10 mm to about 12 mm, or any ranges or volumes therebetween.

In addition, h2 can be about 17.5 mm or less, such as about 16.5 mm or less, such as about 15.5 mm or less, such as about 14.5 mm or less, such as about 13.5 mm or less, such as about 13 mm or less, such as about 8.5 mm or greater, such as about 9.5 mm or greater, such as about 10 mm or greater, such as about 10.5 mm or greater, such as about 11.5 mm or greater, such as about 12 mm or greater, or any ranges or values therebetween. For instance, in one aspect, the lid 110 has a height h2 from about 8.5 mm to about 17.5 mm, such as about 10 mm to about 16.5 mm, such as about 11.5 mm to about 14.5 mm, or any ranges or volumes therebetween. Furthermore, it should be understood that the ranges for h1 and/or h2 provided above, can be measured from the center line 111 or the most distal point of the distal tip 127 as shown in FIGS. 2B (most distal point 127) and 2C (center line 111)

Furthermore, in one aspect, d1 can be about 5 mm or less, such as about 4.9 mm or less, such as about 4.8 mm or less, such as about 4.5 mm or less, such as about 4 mm or less, such as about 3.5 mm or less, such as about 3 mm or less, such as about 2.5 mm or less, such as about 2 mm or less, such as about 1.5 mm or less, such as about 1 mm or more, such as about 2 mm or more, such as about 3 mm or more, such as about 4 mm or more, such as about 4.5 mm or more, or any ranges or values therebetween. For instance, in one aspect, the inner diameter d1 can be from about 1 mm to about 5 mm, such as about 2 mm to about 4.9 mm, such as about 3 mm to about 4.85 mm, or any values or ranges therebetween.

Similarly, in one aspect, d2 can be about 7 mm or less, such as about 35.5 mm or less, such as about 30 mm or less, such as about 25 mm or less, such as about 20 mm or less, such as about 15 mm or less, such as about 10 mm or less, such as about 7.5 m or less, such as about 5 mm or less, such as about 5 mm or more, such as about 5.5 mm or more, such as about 6 mm or more, such as about 6.3 mm or more, such as about 6.4 mm or more, such as about 6.45 mm or more. In one aspect, the outer diameter d2 can be from about 5 mm to about 35.5 mm, such as about 5.5 mm to about 25 mm, such as about 6 mm to about 20 mm, or any ranges or values therebetween. Furthermore, it should be understood that the ranges for d1 and/or d2 provided above, can be measured at the widest, or smallest diameter of the respective d1 and/or d2 in an aspect where the projections 112 are tapered.

In addition, as shown in FIG. 2B, in one aspect, the projections 112 can taper towards a distal end 116 in order to provide further reductions in overspilling without compromising on average wall clearance and evaporation reduction. Thus, in one aspect, one or more projections 112 has a center line 111 which is generally perpendicular to lid 110, and each projection has a taper 113 having an angle relative to the center line 111 of about 2° or less, such as about 1.75° or less, such as about 1.5° or less, such as about 1.25° or less, such as about 1° or less, such as about 0.75° or less, or about 0.25° or greater, such as about 0.4° or greater, such as about 0.65° or greater, or any ranges or values therebetween. For instance, in one aspect, one or more (or each) projections has a taper from about 0.25° to about 2°, such as about 0.35° to about 1.75°, such as about 0.55° to about 1.5°, or any ranges or values therebetween.

Referring next to FIGS. 2D and 2E, where FIG. 2D is a top-down view of FIG. 2A, and FIG. 2E is a view of the cross-section taken along line D-D. Apertures 115 are illustrated adjacent to each radial notch 122. Nonetheless, as shown most clearly in FIG. 2E, the canted distal tip 124 has an angle relative to the plane 119 of the lid 110, which, as shown, may be generally planar. Furthermore, while the plane 119 of the lid 110 and the well base 126 was referred to above as being in parallel planes, as shown in FIG. 2E, it should be understood that, in one aspect, the well base 126 is canted at the same, or similar, angle to the canted distal tip 124. In such a manner, the well base 126 has a consistent thickness (not shown) across its entire cross section. However, as noted above, in one aspect, the well base 126 can be in a plane generally parallel to the plane 119 of the lid, such that the well base 126 has a thickness than increases towards the most distal point of the canted distal tip 124.

Finally, FIG. 2F illustrates a perspective view of the lid 110 of FIG. 2A-2F. as shown, in an aspect having an aperture 115, the aperture 115 may be adjacent to the radial notch 122.

Moreover, as shown in FIG. 3A, in one aspect, the lid 110 can be a single piece having a number of projections 112 equal to a corresponding base 102 (not pictured). Each projection 112 has a radial notch 122 that extends into the at least one sidewall 128 of each projection 112. In addition, the lid 110 contains a frame 132 having a planar portion 134 and a lip 136. The lip 136 can contain one or more angled corners 138, or may have one or more square corners 140. In one aspect, as shown in FIGS. 3A and 3B, two of the corners may be angled in order to provide a reversible locking function to the lid 110 by providing a tighter fit and seal to maintain the lid 110 on the base (not shown) and to further assist with evaporation.

Furthermore, the lip 136 may also have a height h3 that maintains SBS compatibility, but that extends further along the base 102 then prior lids. For instance, the height h3 in the y-direction may be from about 10 mm or more, such as about 10.25 mm or more, such as about 10.5 mm or more, such as about 10.75 mm or more, such as about 11 mm or more, such as about 11.25 mm or more, such as about 11.5 mm or more, up to about 12 mm or less, such as about 11.75 mm or less, such as about 11.7 mm or less, such as about 10 mm to about 12 mm, or about 10.25 mm to about 11.75 mm, such as about 10.5 mm to about 11.7 mm, or any ranges or values therebetween. Namely, the present disclosure has found that lips 136 having heights h3 within the above discussed ranges may further aid in reducing evaporation, which is of heightened importance when using reduced sample sizes.

Further, FIG. 3B illustrates a base 102 that cooperates with the lid 110 of FIG. 3A. As shown, the base 102 contains a support 142 having a skirt 141 that contains a plurality of chambers or wells 104. In the illustrated aspect, the base 102 also contains angled corners 138, which releasably associate with the angled corners 138 of the lid of FIG. 3A. As discussed above, the lid 110 of FIG. 3A may cooperate with each well 104 of FIG. 3B to occupy a portion of the cavity 108 formed by well sidewalls 108. While not shown, it should be understood that when a sample is present, the lid 110 may displace some or all of a gas located between the lid 108 and a sample (not shown), and, in one aspect, may evacuate all of a gaseous headspace contained in each well 104 such that each projection 112 contacts a sample (not shown) contained in the respective well 104.

In addition, FIG. 3C illustrates an assembly 100 according to the present disclosure, where the lid 110 (shown more clearly in FIG. 3A as discussed above) is disposed onto the base 102 (shown more clearly in FIG. 3B above), such that each projection 112 is in reversible association with the respective wells 104 (shown more clearly in FIG. 3B).

FIG. 3D illustrates another aspect of an assembly 100 according to the present disclosure, where the lid 110 is shown in two parts, frame 152 and cover 153. Namely, as shown rows of strips 156 containing projections 112 are releasably affixed to the frame 152. In such a manner, the frame 152 may be formatted to include the correct number of strips 156 for the number of wells 104 contained in the respective base 102. Furthermore, in such an aspect, the cover 153 and/or frame 152 may be formed of any of the substrate materials discussed above, or alternatively, the frame 152 may be formed from a rigid material, such as a metal, which can be aluminum in one aspect. Nonetheless, in one such aspect, the cover 153 may be clear or frosted.

Referring next to FIGS. 4A and 4B, a microtiter plate assembly 200 is shown having a lid 210 having a planar portion 234 and a lip 236. As discussed above, in one aspect, the frame (132 above) may be integral to the lid 210. Furthermore, the lid 210 has a plurality of projections 212 which extend from a proximal end 214 adjacent to the lid 210 to a distal end 216 having a canted distal tip 224. As shown in FIG. 4A, the projections 212 may generally be hollow, and have an external sidewall 246 that contacts the sample, and an internal sidewall 248 that is located on an internal portion of each projection. However, even if the projection is hollow, it should be understood that the radial notch 222 does not penetrate through the external sidewall 246 into the internal sidewall 248. In one aspect, for instance, the projections 212 are hollow as they are formed by injection molding, however, it should be understood that any method known in the art may be used, such as milling.

In addition, FIG. 4A shows more clearly the stop tabs 250. As discussed above, the stop tabs 250 may be used in conjunction with the radial notch 222 and canted distal tip 224 to provide highly accurate height and wall clearances, which will be discussed in greater detail in regards to FIG. 4B. Namely, the stop tabs 250 contact a portion of the base 202 to dispose the projections 212 at specific heights so as to maintain the necessary height and wall clearances (shown more clearly in FIG. 4B). In one aspect, the stop tab 250 may contact a continuous portion of the base 202, or alternatively, may contact a stop tab receiving portion 258. Nonetheless, as shown, the microtiter plate assembly 200 also includes a plurality of wells 244 which are configured to releasably contain the projections 212 in a reversible manner.

Furthermore, as shown more clearly in FIG. 4A, and as discussed above in regards to FIG. 2B, the projections 212 have a height h1 from the stop tab 250 to a distal end 216 and a diameter d1, and the lid 210 has a height h2 from the stop tab 250 height to a well base 226, and a diameter d2. Using these dimensions, the volume of the respective well 204 and the volume of the corresponding projection 212 can be determined to determine the fluid volume displaced by the lid 210.

FIG. 4B contains a close-up depiction of the distal end 216 of a projection 212 of FIG. 3A. Namely, as shown in FIG. 4B, the distal end 216 of the projection 212 has a canted distal tip 224 relative to the well base 226 (e.g. the lower surface of the interior of the well cavity). Furthermore, FIG. 4B more clearly illustrates the sample 252, height clearance 254, wall clearance 256, radial notch 222, apex 251, and radial notch clearance 253 as discussed above. Namely, as shown, the small, but accurate height clearance 254 (e.g. the distance between the most distal portion of the projection 212/portion of the projection 212 closest to the well base 226) is close to the well base 226 to allow sensitive measurements without touching the well base 226 which would interrupt cell growth and monolayer formation. Furthermore, as shown, the wall clearance 256 is very small so as to enable the projection 212 to be removed from the well 244 while still guiding any air bubbles to the radial notch 222.

Furthermore, FIG. 4B shows that the radial notch 222 is located adjacent to the highest portion of the canted distal tip 224 (e.g. the portion of the distal end 216 having the shortest distance from the proximal end 224 or the portion of the canted distal tip 224 furthest from the well base 226). In such a manner, bubbles may be guided by the angle of the canted distal tip 224 to the radial notch 222 so that bubbles may be efficiently evacuated from the sample 252. Further, as shown in FIG. 4B, the sample 252 has a height h that is slightly higher than the sum of the height of the canted portion of the distal tip 224 and the height clearance 254. In such a manner, the sample 252 may extend into the radial notch 222 such that overspilling is prevented but the headspace is fully eliminated.

In addition, FIG. 4C illustrates an aspect of the present disclosure where the projection 212 includes two radial notches 222 and two angled surfaces 225 on the canted distal tip 224, that have a most distal end adjacent to center line C and a highest end adjacent to the respective notch 222. Furthermore, as shown, the projection 212 contains a spotting surface 260, which is generally perpendicular to the well base 226.

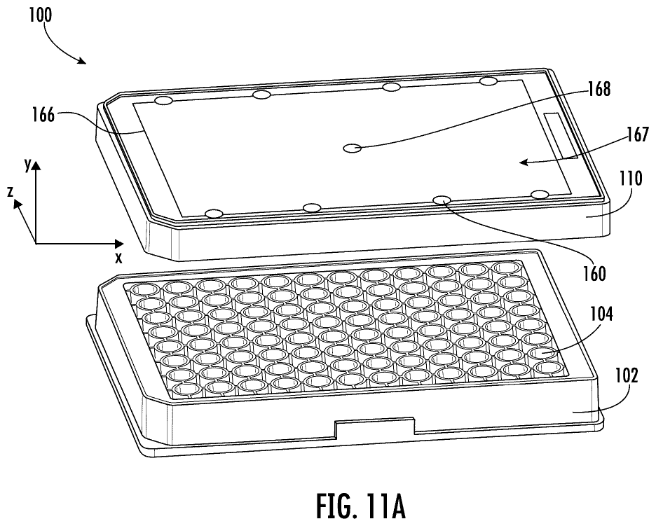

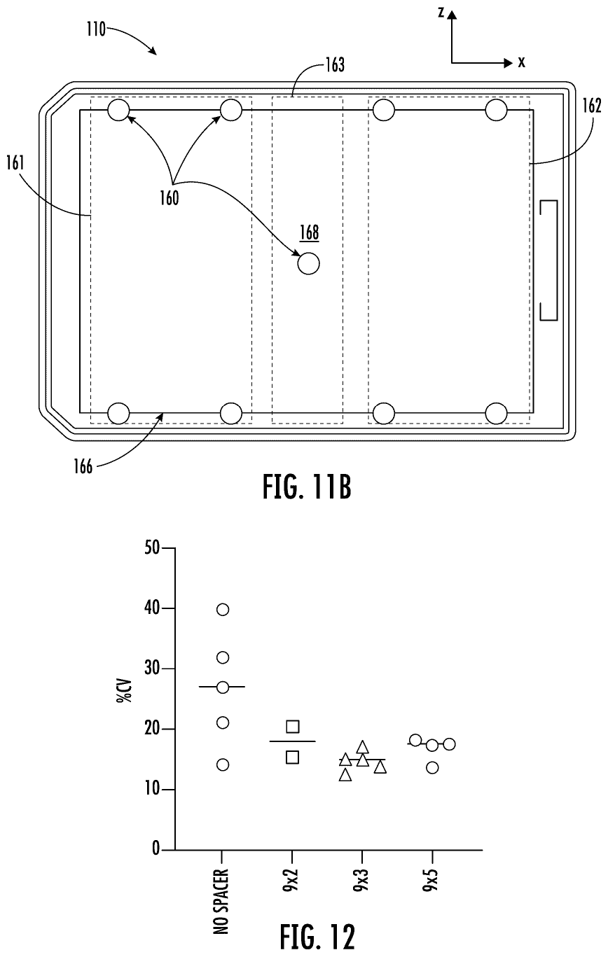

Further, FIGS. 11A and 11B illustrate additional aspects of a base and lid for use in the assembly according to the present disclosure. Namely, as shown in FIG. 11A, a microtiter assembly 100 is illustrated having a base 102 having a plurality of wells 104. Furthermore, FIG. 11A shows a lid 110 having a plurality of projections (not shown) which extend longitudinally from the lid 110 as described above with respect to FIG. 1A. The lid 110 is shown from a top view in FIG. 11B. In particular, the lid 110 can include a plurality of spacers 160 disposed on its exterior surface 167. The spacers 160 can be located about a perimeter 166 of the lid 110, and/or at the center 168 of the lid 110, or in any suitable location. The present inventors have found that the use of such spacers 160 can prevent warping of the lid 110 by increasing downward pressure on the base 102 via the lid 110 to provide more accurate test results and achieve sufficient sample volume consistency across the base 102, leading to more consistent oxygen depletion rates across a full base 102.

As shown in FIG. 11B, multiple spacers 160 can be present within a first outer region 161 of the lid 110, multiple spacers 160 can be present within a second outer region 162 of the lid 110, and a single spacer 160 can be located within a central region 163 of the lid 110. However, it is to be understood that additional spacers 160 can be located within any of the regions 160, 161, or 162 to achieve reduced warpage of the lid 110. Further, the spacers 160 can be injection molded during the formation of the lid 110 and can be formed from the same material as the rest of the lid 110.

Moreover, it should be understood that the spacers 160 can have varying thicknesses in the y-direction in order to provide uniformity and reduced warpage of the lid 110. However, in other embodiments, the spacers 160 can each have the same thickness. For instance, in some embodiments, the spacers 160 can each have a thickness ranging from about 100 micrometers to about 500 micrometers, such as from about 125 micrometers to about 475 micrometers, such as from about 150 micrometers to about 450 micrometers.

In one particular embodiment, the spacers 160 in the central region 163 can have a thickness that is greater than the thickness of the spacers 160 in the first outer region 161 and the second outer region 162 to provide increased downward pressure at the center of the lid 110 where more warpage can occur. In such an embodiment, the spacers 160 in the central region can have a thickness ranging from about 175 micrometers to about 500 micrometers, such as from about 200 micrometers to about 475 micrometers, such as from about 225 micrometers to about 450 micrometers, while the spacers 160 in the first outer region 161 and the second outer region 162 can have a thickness ranging from about 100 micrometers to about 250 micrometers, such as from about 125 micrometers to about 225 micrometers, such as from about 150 micrometers to about 200 micrometers.

As shown in FIG. 12, a comparison of the coefficient of variation of oxygen depletion measurements in the wells of a lid 110 of the microtiter plate assembly 100 of FIGS. 11A and 11B utilizing spacers 160 having various thicknesses compared to a lid 110 with no spacers shows that the coefficient of variation (% CV) is reduced when spacers 160 are utilized compared to when no spacers are utilized.

Aspects of the present disclosure may be further understood according to the following nonlimiting examples.

### Example 1

Lids according to the present disclosure were formed and positioned into the respective wells by allowing the projections to sink into the respective well and then pushing the lid into place, allowing controlled bubble dissipation. Oxygen ingress was tested to show re-oxygenation of fully de-oxygenated samples by perfusing the samples with nitrogen gas, monitoring deoxygenation levels using dOxyBeads® or OpTech by Mocon, or an oxygen sensitive phosphorescent probe (MitoXpress® Xtra), and measured using a fluorescence plate reader (CLARIOstar BMG Labtech, where reader software reports probe emission intensity or lifetime as defined above). To maintain sample deoxygenation, plate preparation was conducted in a hypoxia bag flushed and filled with nitrogen gas. A negative (100% air saturation) and positive (0% O₂) control were added as well as a sample containing an oil layer in the headspace.

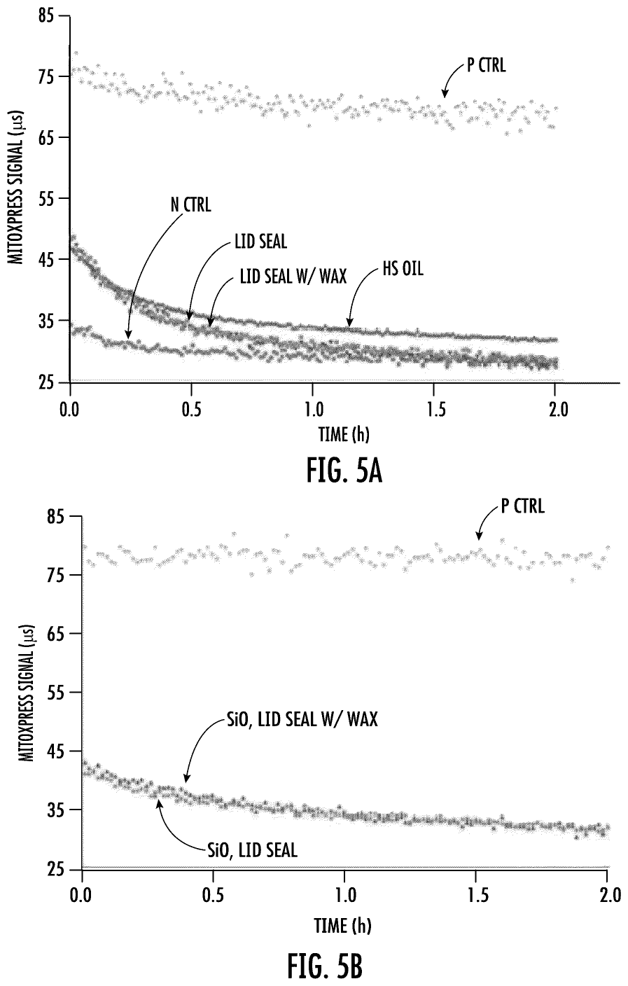

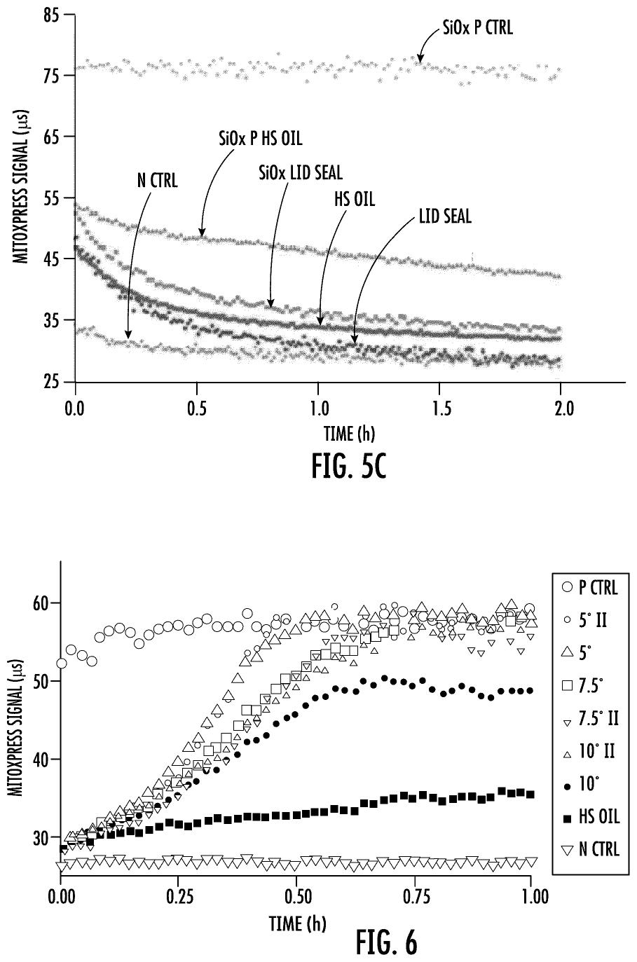

As shown in FIG. 5A, two samples having lid-seals/lids according to the present disclosure were tested, where one sample had the radial notch filled with paraffin wax. As illustrated in FIG. 5A, the lid with wax exhibited similar re-oxygenation as to the lid without wax, showing that radial notches having a radial cross section of 1.75 mm² and a canted distal end angled at 5° play no significant role in oxygen back diffusion. Similarly, FIG. 5B contains two samples prepared according to FIG. 5A except that the well and base included a further glass coating. FIG. 5B illustrates that the radial notch does not significantly contribute to oxygen back diffusion even when the microplate wells were coated with a glass coating to reduce re-oxygenation from the lid and well substrate.

Furthermore, FIG. 5C shows a comparison of oxygen ingress from both coated and uncoated microplate wells. As shown in FIG. 5C, lids formed according to the present disclosure exhibited low rates of back diffusion of oxygen. Namely, as shown in FIGS. 5A-5C, the samples formed according to the present disclosure effectively dissipated air bubbles and had back diffusion comparable to the oil-in-headspace samples.

### Example 2

Lids according to the present disclosure were formed that had a radial notch having a radial cross section of 1.75 mm² and canted distal ends angled at 5°, 7.5° and 10°. Each of the inventive samples was measured in two separate runs using A549 cells to test for oxygen respiration. FIG. 6 illustrates oxygen consumption by A549 cells seeded at a density of 65,000 cells and a fill volume of 50 μL. As shown in FIG. 6, the lids formed according to the present disclosure were more sensitive to respiration showing a 5-fold increase in rate of probe signal change as compared to the oil in headspace sample, and showed full deoxygenation within the target time of 45 minutes (completed in 30 minutes as shown, which was within the 45 minute target).

### Example 3

A sample according to the present disclosure was formed having the following dimensions:

Parameters

| A: Wall clearance | 0.05 | mm |
|---|---|---|
| B: Height clearance | 0.05 | mm |
| C: Pillar Angle | 5* | |
| D: Notch size | 1.75 | mm² |
| Sample volume | 10.09 | μL |
| Fill volume | 50 | μL |

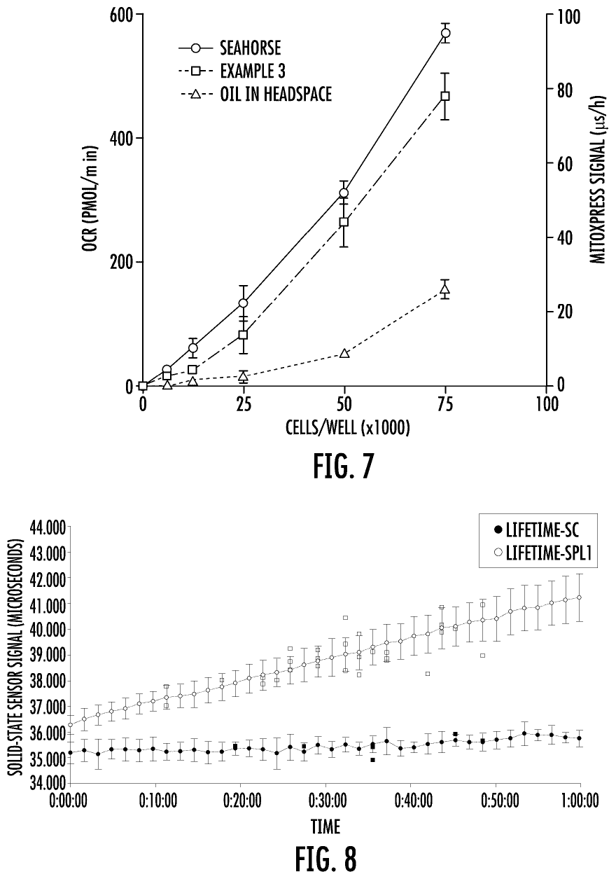

As shown in FIG. 7, the examples according to the present disclosure were benchmarked against an oil-in-headspace control as well as an XF flux Analyzer. As shown in FIG. 7, the lid according to the present disclosure exhibited similar sensitivity to the Seahorse analyzer, and much greater sensitivity as compared to the oil-in-headspace sample, even at very low seeding densities.

### Example 4

Interrogation was conducted in the same manner to Example 1 above except that a solid state sensor of an oxygen-sensitive photoluminescent dye contained in an oxygen permeable carrier matrix was used that was deposited on the canted distal tip of the projection. As described above, the sample was interrogated using a plate reader (BioTek Cytation 5 from Agilent Technologies). Cell samples of 40,000 A549 cells per well were used in F12K media. As shown in FIG. 8, solid state sensors deposited on a lid according to the present disclosure exhibited good oxygen depletion sensing over the 1 hour measurement with a mean rate of 5.89 μs/h and a % CV of 12.82.

### Example 5

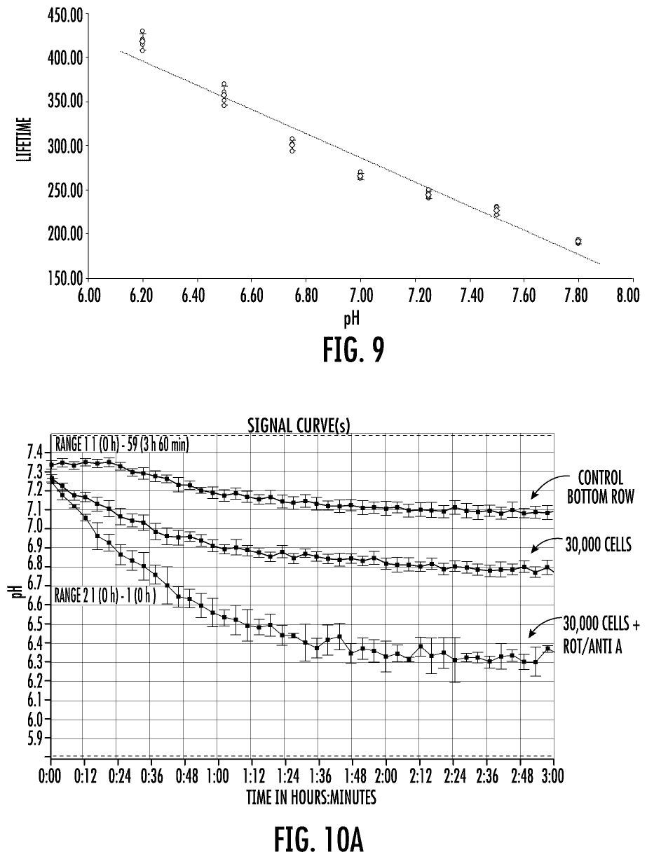

Interrogation was conducted in the same manner to Example 1 above except that both change in pH and oxygen depletion were measured. Measurements were conducted on a BioTek Cytation 5 and BMG Clariostar plate reader as discussed above. pH calibration was obtained as shown in FIG. 9, illustrating compatibility with a lid according to the present disclosure.

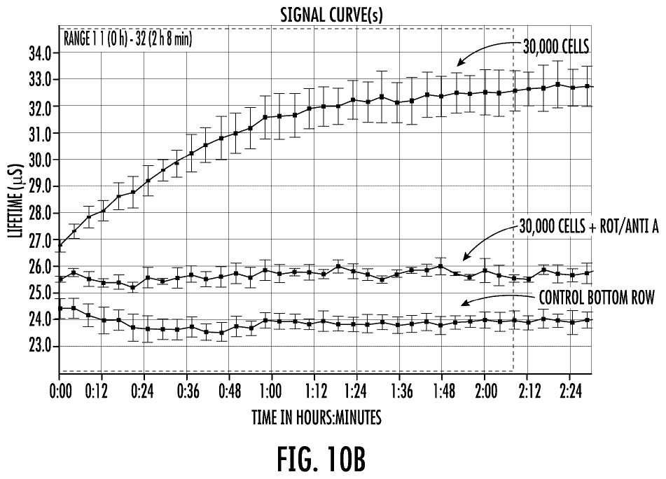

Cell samples of 30,000 A549 cells, rotenone, antimycin A, and XF DMEM media with 5 mM HEPES were used in conjunction with a lid and process according to Example 1. Both pH and oxygen depletion were measured simultaneously in the samples. As shown in FIG. 10A (pH) and FIG. 10B (oxygen depletion), the lid of the present disclosure exhibited sensitive oxygen depletion measurements during multiplexing, as illustrated with pH in this example.

### Example 6

Cell samples of 30,000 A549 cells were plated overnight and then treated with tamoxifen for either 1 hour or 24 hours in the plate assembly of the present disclosure prior to assaying the cells to determine oxygen consumption levels and cell viability. Cells were loaded with calcein AM and hoescht dye pre-assay. Cell culture media was replaced with media containing MitoXpress Xtra, then cells were imaged using a GFP LED/Filter set using a Cytation 5. The MitoXpress Xtra assay was initiated by inserting the lid of the present disclosure onto the base of the plate assembly.

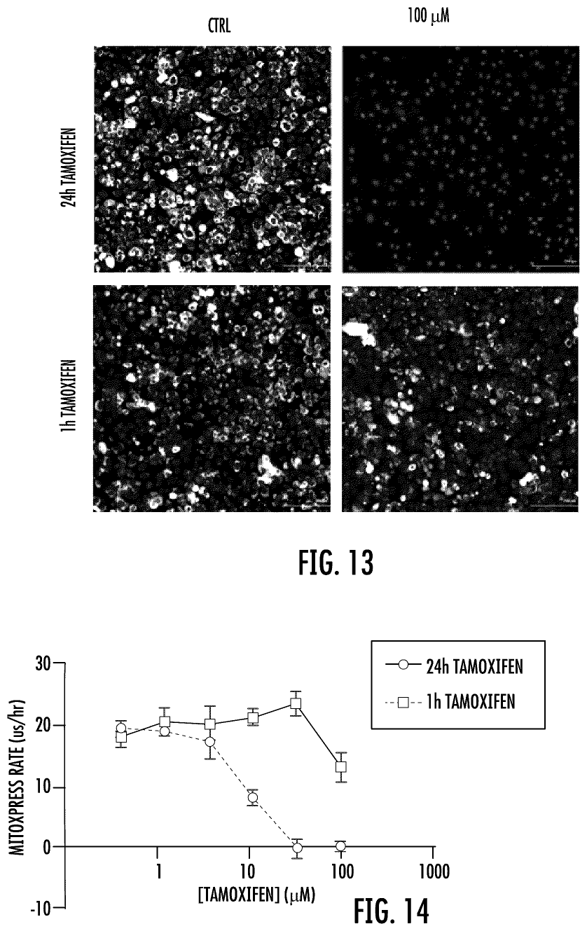

As shown in FIGS. 13-14, the ability to multiplex cell viability and mitochondria respiration measurements was demonstrated and adds context to data. It allows a user to determine if a compound or drug is directly affecting the mitochondria as mitochondrial inhibitors will be revealed by an acute decrease in oxygen consumption or if the effect is due to broad cytotoxicity. Loss of viability is revealed by Calcein AM. When cells are treated for 24 h, the assay can also be used to determine the overall metabolic flexibility of cells. If respiration is decreased but viability remains, then the cells can adapt to produce ATP via glycolysis in addition to OXPHOS.

### Example 7

Cells were loaded with MitoTracker and hoechst as per manufactures instructions. Cells were washed with media then the media was replaced with media containing MitoXpress Xtra. Imaging was carried out as the plate was incubating with compounds using a Cytation 5 with a RFP LED/Filter set. Then, the lid of the present disclosure was placed onto the base of the plate assembly and MitoXpress Assay was run as normal.

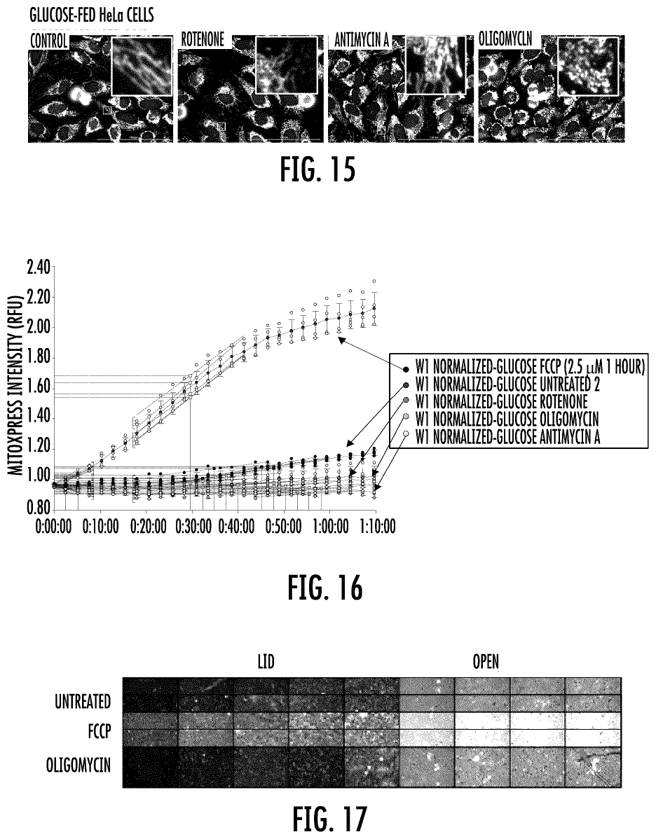

FIG. 15 shows the effects of compounds on the mitochondrial network. Rotenone, Antimycin and Oligomycin cause the network to condense, and divide compared to the control. The effects in structure are then reflected in the rates of oxygen consumption showing a direct correlation between structure and function. Different compounds lead to different effects. Some promote fusion, some inhibit fusion, some fission some limit fission. The novel insight with this method is to correlate changes in structure of the mitochondria to functional changes in respiration as shown in FIG. 16 in the same well for the first time.

### Example 8

Cells were loaded with 2.5 μM JC-1 and hoechst for 45 minutes. Cells were washed 3× in media then initial images were acquired using a GFP and RFP LED/Filter set using a Cytation 5. Media was replaced to media containing MitoXpress Xtra. Cells were treated with compounds for 1 hour then imaged as shown in FIG. 17. The respiration assay was started by inserting the lid of the present disclosure onto the base of the plate assembly. Cells were measured for oxygen consumption using a MitoXpress Xtra filter cube as well as 2 monochromator measurements (Red 475ex, 535 em and Green 475 ex 590 em). The JC-1 ratio was calculated by dividing red FI/green FI.

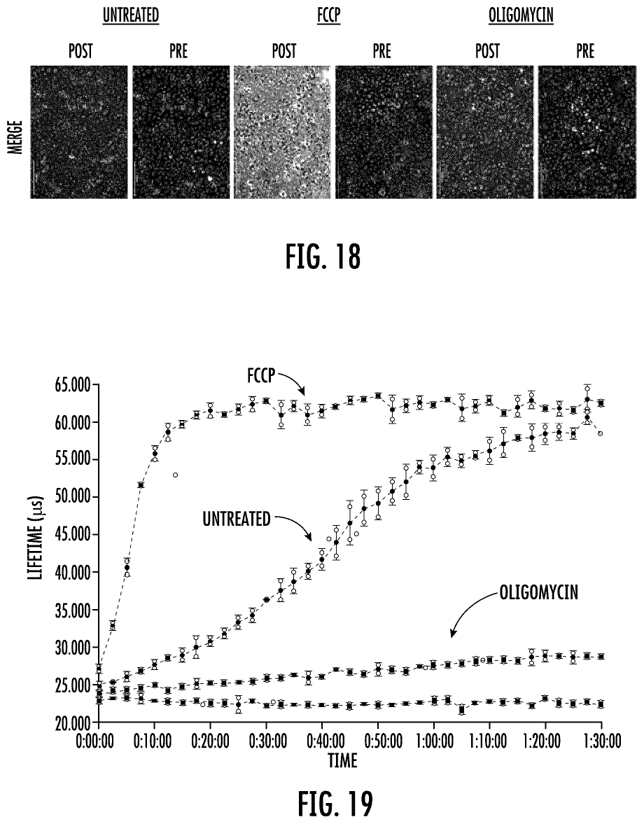

The simultaneous measurement of mitochondrial membrane potential (MMP) and oxygen consumption from the same well was demonstrated. FIGS. 17 and 18 show that the traditional method of measuring JC-1 using open well measurement is comparable to when the lid is in the well. There is an improvement under the lid as background signal is decreased. The fluorescence images in FIG. 18 show pre-treatment and post-treatment JC-1plus hoechst staining in live cells. FIG. 19 shows oxygen consumption traces and rate data from compound treated cells. FIG. 20 shows kinetic JC-1 data reporting on MMP using PMT measurements of JC-1 ratios from the same wells as the MitoXpress Xtra data. The controls responded as expected. The assay was able to detect changes in hyperpolarization, depolarization, and basal levels as well as relating changes in MMP to changes in mitochondrial respiration rates. The method can distinguish between loss of membrane potential due to uncoupling and due to inhibition of the electron transport chain, which is otherwise undetectable.

### Example 9

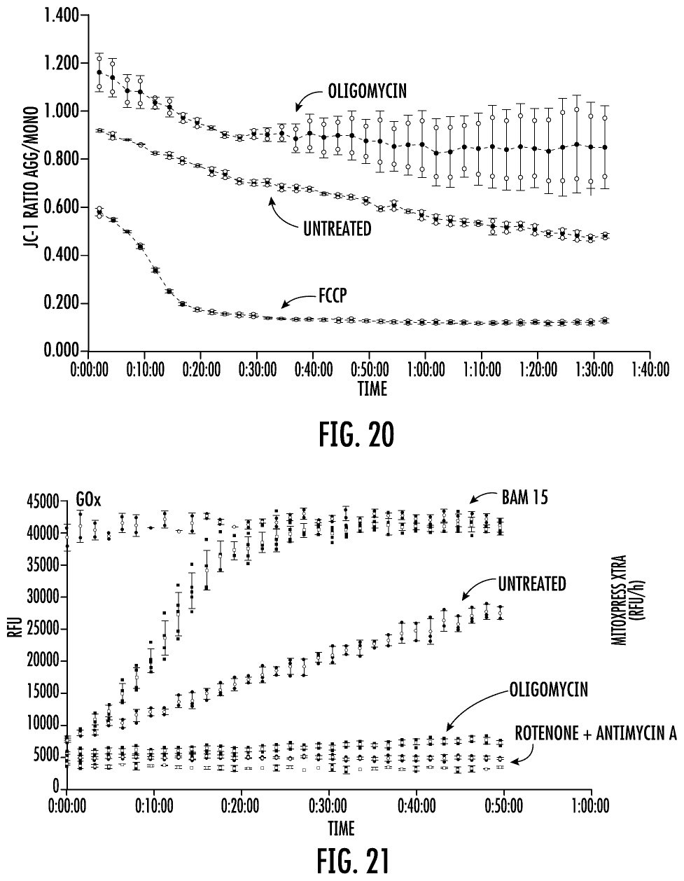

Cells were counted and resuspended in media with MitoXpress Xtra reagent. They were then plated on to a PDL-coated microplate prior to a centrifugation step. Cells were treated with compounds before inserting the lid of the present disclosure and starting the measurement in the plate reader. FIG. 21 shows that THP-1 cells grown in suspension are responding to control compounds as expected. This validates the method for attaching the cells to the PDL plate and that MitoXpress® Xtra is suitable for measuring respiration.

### Example 10

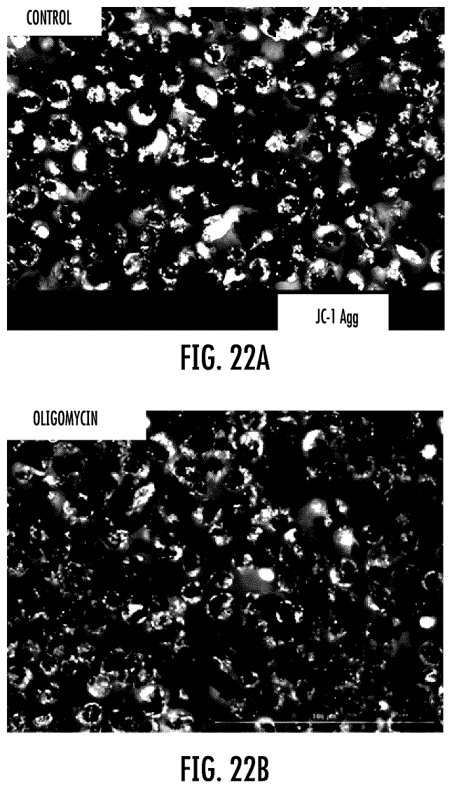

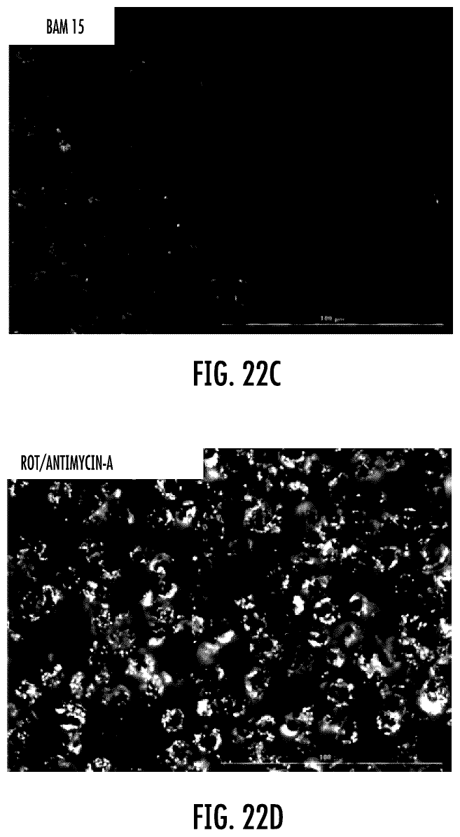

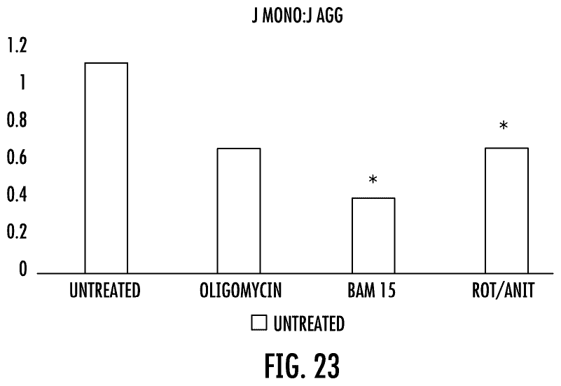

Cells were loaded with 2.5 μM JC-1 and Hoechst for 45 min. Cells were washed 3× in media then initial images were acquired using a GFP and RFP LED/Filter set using a Cytation 5. The JC-1 Ratio shown in FIG. 23 was calculated by dividing red FI/green FI. As with the adherent cell assay, suspension cells can also be loaded with JC-1 and imaged. Cells responded to control compounds as expected. It was possible to image cells post the MitoXpress Xtra assay without disturbing the monolayer as shown in FIGS. 22A-22D.

These and other modifications and variations to the present disclosure may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A microtiter plate assembly comprising:
a base comprising at least one well; and
a lid comprising at least one translucent projection corresponding with at least one well;
wherein the at least one translucent projection extends from a proximal end adjacent to the lid to a distal end having a radial notch and a canted tip, the canted tip having an angle of about 5° or greater relative to a plane of the lid; and
wherein a wall clearance is defined between a sidewall of the at least one projection and a sidewall of the at least one corresponding well, and wherein a radial notch clearance is defined between an apex of the radial notch and the sidewall of the corresponding well, wherein the wall clearance is 50% or less than the radial notch clearance.

2. The microtiter plate assembly as defined in claim 1, wherein at least a portion of the assembly is formed from a substrate material having an oxygen transmission rate of about 600 cm³/m²/24 hours or less, and/or wherein at least a portion of the assembly is coated with a polymeric or ceramic compound having an oxygen transmission rate of about 120 cm³/m²/24 hours or less.

3. The microtiter plate assembly as defined in claim 1, wherein at least a portion of the assembly is coated with a Al₂O₃, SiO₂, silicon oxide, silicon nitride, silicon oxynitride, aluminum oxide, Al₂O₃ alternated with poly-acrylate, or a combination thereof.

4. The microtiter plate assembly as defined in claim 1, wherein the radial notch has a radial cross section of about 0.7 mm² or greater.

5. The microtiter plate assembly as defined in claim 1, wherein the assembly is configured to measure oxygen consumption of fluid sample having a volume of 100 μL or less.

6. The microtiter plate assembly as defined in claim 1, wherein the lid is in reversible association with the base.

7. The microtiter plate assembly as defined in claim 1, wherein a radius of the radial notch has a length of about 5% to about 30% of a length of a radius of a cross section of the projection including the radial notch, at the proximal end of the projection, the distal end of the projection, or both the proximal end and the distal end of the projection.

8. The microtiter plate assembly as defined in claim 1, wherein the at least one projection has a proximal end radius and a distal end radius, wherein the distal end radius is about 2% to about 15% less than the proximal end radius.

9. The microtiter plate assembly as defined in claim 1, wherein the assembly is configured to measure oxygen depletion of a sample having a seeding density of 250,000 cells or less in 45 minutes or less.

10. The microtiter plate assembly as defined in claim 1, wherein the lid is milled, extruded, injection molded, or a combination thereof.

11. The microtiter plate assembly as defined in claim 1, wherein the assembly includes from about 1 to about 384 wells and about 1 to about 384 corresponding projections.

12. The microtiter plate assembly as defined in claim 1, wherein the lid is a single piece.

13. The microtiter plate assembly as defined in claim 1, wherein the lid is formed from a frame and one or more rows containing a plurality of translucent projections attached to the frame.

14. The microtiter plate assembly as defined in claim 13, wherein the lid contains eight rows each row containing twelve corresponding projections.

15. The microtiter plate assembly as defined in claim 1, wherein at least a portion of the assembly is formed from an acrylate polymer.

16. The microtiter plate assembly as defined in claim 1, wherein the lid includes a plurality of spacers.

17. A microtiter plate assembly comprising:
a base comprising a plurality of wells spaced apart in an array, where each well is adjacent to a second well; and
a lid comprising a plurality of translucent projections spaced apart in an array corresponding with the array of the plurality of wells, where each projection is adjacent to a second projection;
wherein the plurality of translucent projections extend from a proximal end adjacent to the lid to a distal end having a radial notch and a canted tip, the canted tip having an angle of about 5° or greater relative to a plane of the lid; and
wherein a height clearance is defined between a most distal point of the distal end of each projection and a well base of the respective well, and wherein a percent variation of the height clearance between the projection and the well and the adjacent second projection and second well is about 10% or less.

18. A method of measuring an oxygen depletion rate utilizing the assembly of claim 1 comprising:
placing a sample and an oxygen-sensitive probe into the at least one well,
contacting the sample with the corresponding projection, and
measuring the oxygen depletion with a fluorescence plate reader.

19. The method as defined in claim 18, the method further comprising measuring acidification.

20. The method as defined in claim 18, wherein the oxygen-sensitive probe is selected from the group consisting of a solid state sensor coated on the distal end of the projection, a soluble sensor, a particulate sensor, a planar sensor, one or more sensor spots on the lid and microplate, or combination thereof and wherein the sample has a volume of 100 μL or less, preferably wherein the sample volume is 70 μL or less.
